(19) United States
(12) Patent Application Publication
(10) Pub. No.: US 2014/0161796 A1
(43) Pub. Date: Jun. 12, 2014

(54) SINGLE CHAIN PROTEINS WITH C-TERMINAL MODIFICATIONS

(71) Applicants: Andreas LOEW, Somerville, MA (US); Hilmar EBERSBACH, Basel (CH)

(72) Inventors: Andreas LOEW, Somerville, MA (US); Hilmar EBERSBACH, Basel (CH)

(21) Appl. No.: 14/026,599

(22) Filed: Sep. 13, 2013

Related U.S. Application Data
(60) Provisional application No. 61/700,529, filed on Sep. 13, 2012.
(60) Provisional application No. 61/789,856, filed on Mar. 15, 2013.

(51) Int. Cl. G01N 33/53; C07K 16/18

(52) U.S. Cl. CPC G01N 33/5306; C07K 16/18
USPC 424/133.1; 530/387.3; 536/23.53; 435/320.1; 435/252.33; 435/7.94; 435/252.31; 435/252.34; 435/252.3; 435/254.2; 435/254.21; 435/254.23; 435/328

(57) ABSTRACT

The invention pertains to an isolated V_{H} single domain antibody comprising a C-terminal modification, where the C-terminal modification comprises a deletion of at least one amino acid residue that eliminates the interaction of a pre-existing antibody with the single domain antibody without interfering with the binding of the single domain antibody with its target.

### RELATED APPLICATIONS

This application claims priority to U.S. Provisional Application No. 61/700,529 filed 13 Sep. 2012 and 61/789,856 filed 15 Mar. 2013, the contents of which are incorporated herein by reference in their entireties.

### BACKGROUND OF THE INVENTION

Circulating pre-existing antibodies may arise from incidental or occupational exposure to foreign protein, use of antibodies as therapeutic agents following infection or vaccination, or for unknown reasons.

These pre-existing antibodies give rise to erroneous results inconsistent with the patient's clinical picture. The interference is variable, complex and unpredictable because of the wide range of affinities and avidities among the various endogenous antibodies, or both. Pre-existing antibodies are not only difficult to recognize, but are problematic to eliminate. Defining the precise mechanisms of interference by pre-existing antibodies has been challenging because of variation in the phenomena produced by the antibodies. Pre-existing antibodies may increase readings in some assays but decrease the results in others. Pre-existing antibodies may be identified by nonlinearity in some assays but show perfect linearity on serial dilution in others. Interference from some antibodies may be blocked by commercially available “blocking reagents”, but interferences from other antibodies are not.

Accordingly, a need exists to produce a binding molecule that bind to the desired target of interest, but with reduced or no interaction with pre-existing antibodies.

### SUMMARY OF THE INVENTION

The invention is based on the surprising discovery that pre-existing antibodies in human sera bind to human single domain antibodies and produce a pre-existing immune response. Even more surprising and unexpected, is the finding that modifications at the C-terminal region of a human V_{H} single domain antibody, eliminates the pre-existing immune response. Based on the data disclosed herein, there is no evidence however, that there is a pre-existing immune response to the C-terminus of a human V_{L} single domain antibody.

Accordingly, in one aspect, the invention pertains to an antigen binding domain (ABD) comprising a C-terminal modification, wherein the C-terminal modification comprises the addition or deletion of at least one amino acid residue such that the addition or deletion of at least one amino acid residue to the ABD eliminates the interaction of a pre-existing antibody with the ABD without interfering with the binding of the ABD with its target.

In another aspect, the invention pertains to an antigen binding domain (ABD) comprising a C-terminal modification, wherein the C-terminal modification comprises the deletion of at least one amino acid residue such that the deletion of at least one amino acid residue from the ABD eliminates the interaction of a pre-existing antibody with the ABD without interfering with the binding of the ABD with its target.

In another aspect, the invention pertains to an isolated V_{H} single domain antibody comprising a C-terminal modification, wherein the C-terminal modification comprises a deletion of at least one amino acid residue such that the deletion of at least one amino acid residue in the single domain antibody eliminates the interaction of a pre-existing antibody with the single domain antibody without interfering with the binding of the single domain antibody with its target.

In one embodiment, the C-terminal of the isolated single domain antibody is exposed such that the exposed C-terminal is available for interaction with the pre-existing antibody, and wherein the C-terminal modification reduces the exposure of the C-terminal to the pre-existing antibody.

In one embodiment, the C-terminal modification modifies the C-terminus of the isolated single domain antibody by a mechanism selected from the group consisting of eliminating the interaction of the pre-existing antibody by altering the three dimensional configuration of the C-terminal single domain antibody such that the pre-existing antibody no longer recognizes the single domain antibody, alters the exposure of the C-terminal single domain antibody to the pre-existing antibody, alters the steric hindrance between the single domain antibody and the pre-existing antibody, disrupts at least one conformational neoepitope in the C-terminus, and shields at least one neoepitope in framework of the single domain antibody.

In one embodiment, the single domain antibody is a human V_{H}.

In one embodiment, the human V_{H} is selected from the group consisting of SEQ ID NO: 8, SEQ ID NO: 9, SEQ ID NO: 10, SEQ ID NO: 11, SEQ ID NO: 12, and SEQ ID NO: 13.

In one embodiment, the C-terminal modification further comprises the deletion of at least one additional amino acid residue from the C-terminus of the single domain antibody.

In one embodiment, the C-terminal modification further comprises the deletion of at least two additional amino acid residues from the C-terminus of the single domain antibody.

In one embodiment, the C-terminal modification further comprises the deletion of at least three, at least four, at least five, at least six, at least seven, at least eight, at least nine additional amino acid residues from the C-terminus of the single domain antibody.

In one embodiment, the C-terminal modification is the deletion of an amino acid sequence selected from the group consisting of Arg, Arg-Thr, Arg-Thr-Val, Gly-Gln, and Gly-Gln-Pro.

In one embodiment, the C-terminal modification eliminates a pre-existing antibody response at least by about 10% compared with a single domain antibody without the C-terminal modification.

In one embodiment, the isolated single domain antibody comprises a V_{H} comprising SEQ ID NO: 8 and the C-terminal modification comprises a deletion of one amino acid to three amino acids. In one embodiment, the isolated single domain antibody comprises a V_{H} comprising SEQ ID NO: 9 and the C-terminal modification comprises a deletion of one amino acid to three amino acids. In one embodiment, the isolated single domain antibody comprises a V_{H} comprising SEQ ID NO: 10 and the C-terminal modification comprises a deletion of one amino acid to three amino acids. In one embodiment, the isolated single domain antibody comprises a V_{H} comprising SEQ ID NO: 11 and the C-terminal modification comprises a deletion of one amino acid to three amino acids. In one embodiment, the isolated single domain antibody comprises a V_{H} comprising SEQ ID NO: 12 and the C-terminal modification comprises a deletion of one amino acid to three amino acids. In one embodiment, the isolated single domain antibody comprises a V_{H} comprising SEQ ID NO: 13 and the C-terminal modification comprises a deletion of one amino acid to three amino acids.

In another aspect, the disclosure pertains to a nucleic acid encoding a composition the V_{H} single domain antibody comprising a C-terminal modification, wherein the C-terminal modification comprises a deletion of at least one amino acid residue such that the deletion of at least one amino acid residue from the single domain antibody eliminates the interaction of a pre-existing antibody with the single domain antibody without interfering with the binding of the single domain antibody with its target; an expression vector comprising the nucleic acids; and a host cell or organism comprising the expression vector.

In another aspect, the disclosure pertains to a pharmaceutical composition comprising a V_{H} single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target.

In another aspect, the disclosure pertains to a method of eliminating a pre-existing immune response in a subject comprising: administering a single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target.

In another aspect, the disclosure pertains a method of improving a response to single domain antibody in a subject having a pre-existing antibody against single domain antibody, comprising: administering single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target.

In another aspect, the invention pertains to a method of predicting whether a pre-existing antibody will produce a pre-existing immune response with a single domain antibody comprising: contacting the single domain antibody with a human sample; determining whether a pre-existing antibody, if present in the human sample, binds to the single domain scaffold; and modifying the C-terminal region of single domain antibody by the deletion of at least one amino acid residue such that the C-terminal modification eliminates the interaction of the pre-existing antibody with the single domain antibody. In one embodiment, the human sample is selected from the group consisting of blood and serum.

### BRIEF DESCRIPTION OF THE DRAWINGS

FIG. 1 shows the cleavage positions in an antibody (positions A, B, C) that may result in an exposed C-terminus which causes a response to pre-existing antibodies;

FIG. 2A-2B illustrates examples of antibodies that may comprise exposed C-termini. 2A is a monoclonal antibody and a Fab-(Fab) and a Fv-fragment (Fv) and a single chain antibody (scFv); and 2B is a single domain antibody (VH and VL);

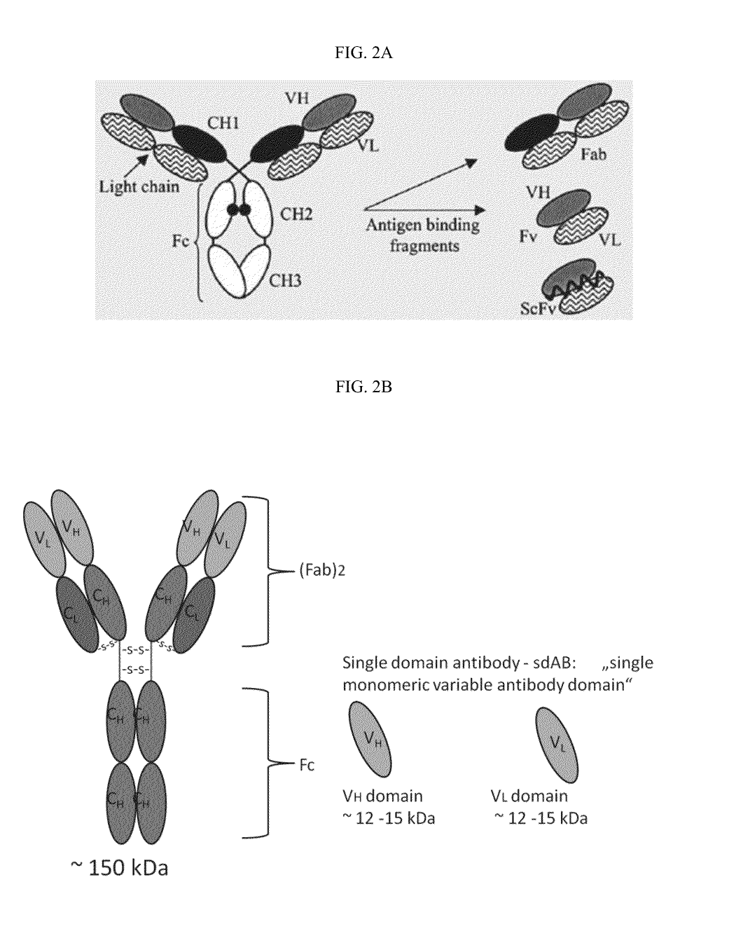

FIG. 3 shows the pre-existing response to three human single domain V_{H} scaffolds and three human V_{L} scaffolds;

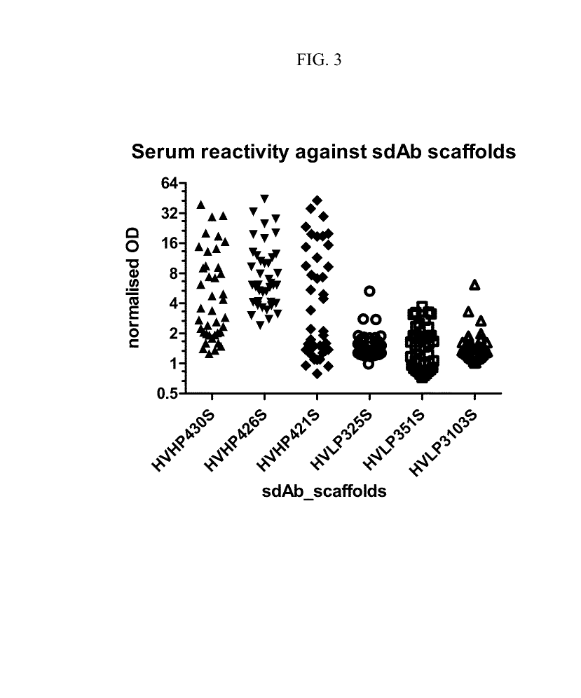

FIG. 4 shows the pre-existing response in human sera with VH-HVHP426 single domain scaffold with various C-terminal amino acid additions;

FIG. 5 shows the pre-existing response in human sera with VH-HVHP420 single domain scaffold with various C-terminal amino acid additions;

FIG. 6 shows the pre-existing response in human sera with VH-HVHM81 single domain scaffold with various C-terminal amino acid additions;

FIG. 7 shows the pre-existing response in human sera with VH stabilized S-S VH-HVHP421S single domain scaffold with various C-terminal amino acid additions;

FIG. 8 shows the pre-existing response in human sera with VH stabilized S-S VH-HVHP430S single domain scaffold with various C-terminal amino acid additions;

FIG. 9 shows the pre-existing response in human sera with VH stabilized S-S VH-HVHP426S single domain scaffold with various C-terminal amino acid additions;

FIG. 10 shows the response in human sera with VL-HVLP335 single domain scaffold with various C-terminal amino acid additions;

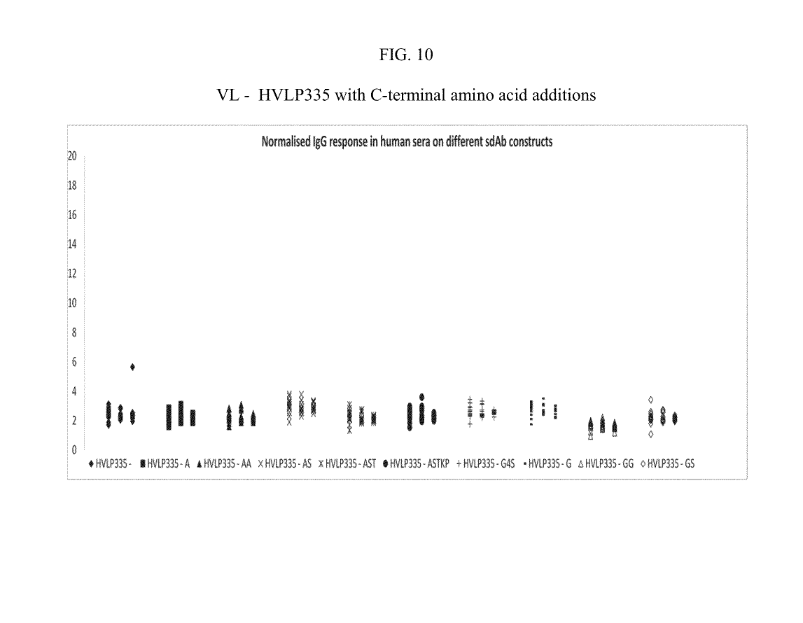

FIG. 11 shows the response in human sera with VL-HVLP325 single domain scaffold with various C-terminal amino acid additions;

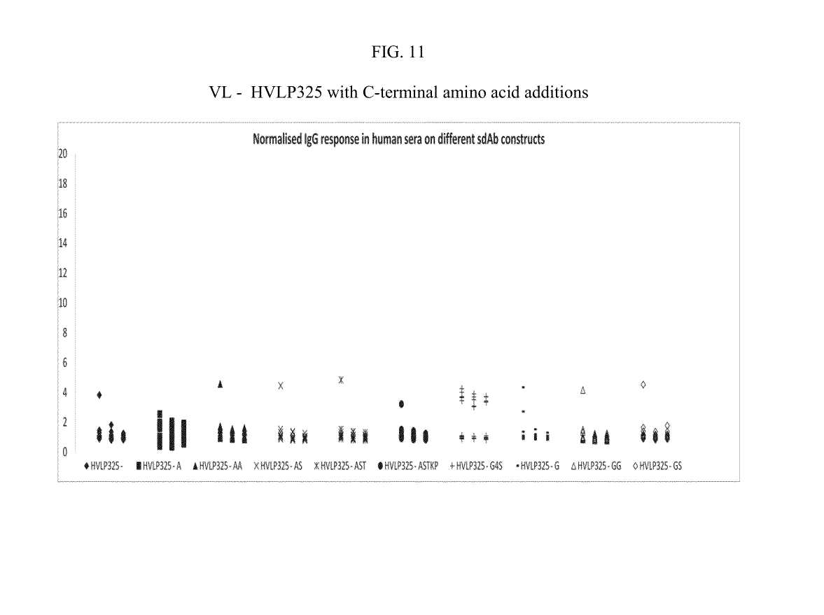

FIG. 12 shows the response in human sera with VL-HVLP351 single domain scaffold with various C-terminal amino acid additions;

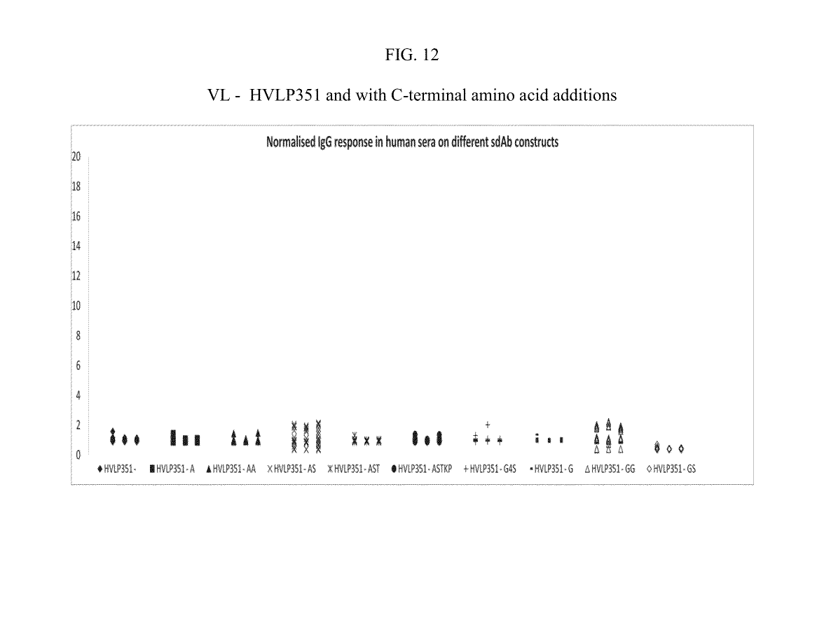

FIG. 13 shows the response in human sera with VL stabilized S-S HVLP3103S single domain scaffold with various C-terminal amino acid additions;

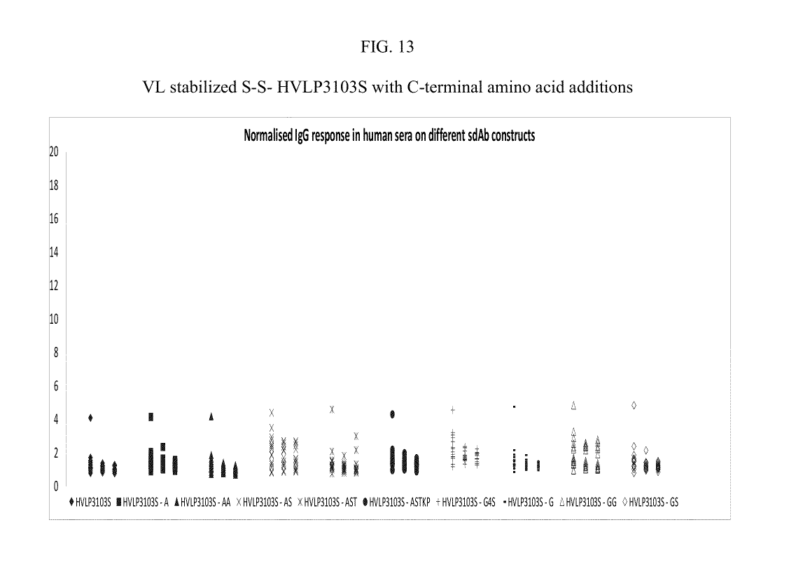

FIG. 14 shows the response in human sera with VL stabilized S-S HVLP325S single domain scaffold with various C-terminal amino acid additions;

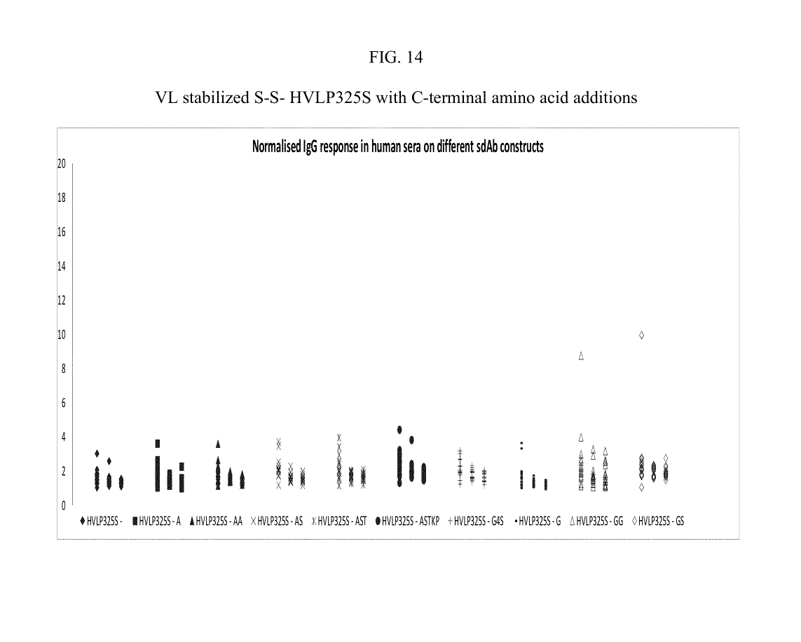

FIG. 15 shows the response in human sera with VL stabilized S-S HVLP351S single domain scaffold with various C-terminal amino acid additions;

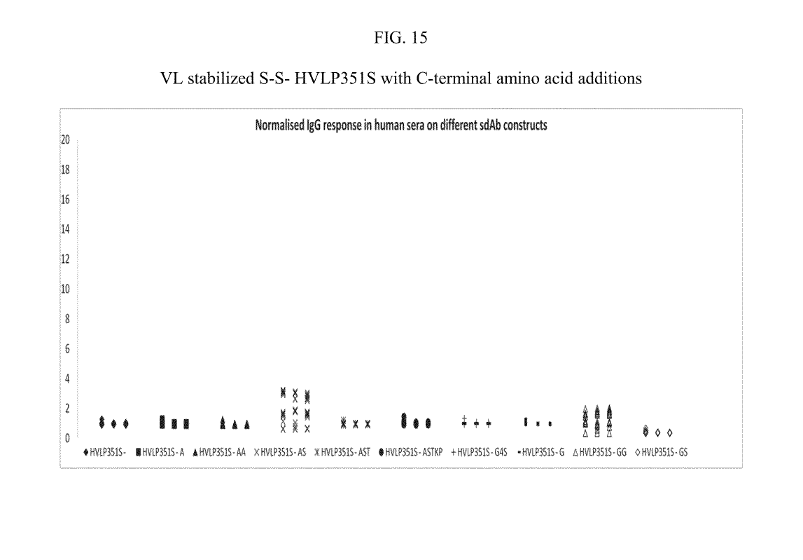

FIG. 16 shows the response in human sera with VL-VL stabilized S-S single domain scaffolds HVLP335, HVLP3103S, and HVLP325 with C-terminal amino acid deletions;

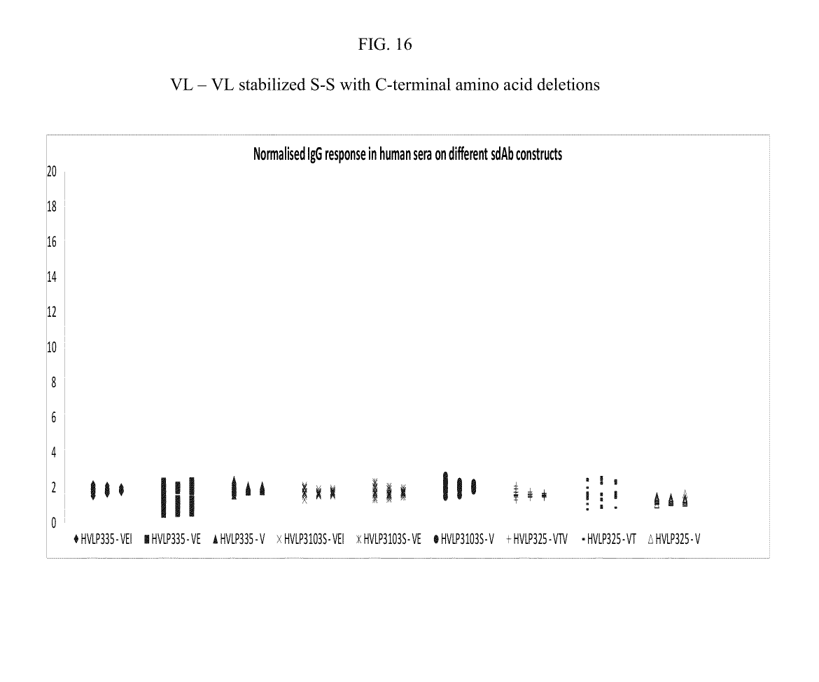

FIG. 17 shows the response in human sera with VL-VL stabilized S-S with single domain scaffolds HVLP325S, and HVLP351 and HVLP351S with C-terminal amino acid deletions;

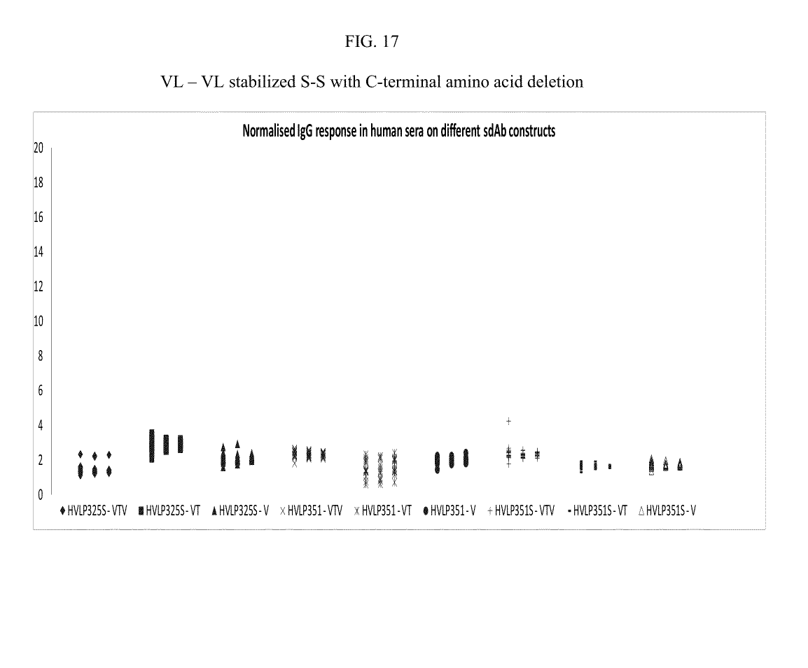

FIG. 18 shows the response in human sera with VH-VH stabilized S-S with single domain scaffolds HVHP426, HVHP426S and HVHP420 with C-terminal amino acid deletions;

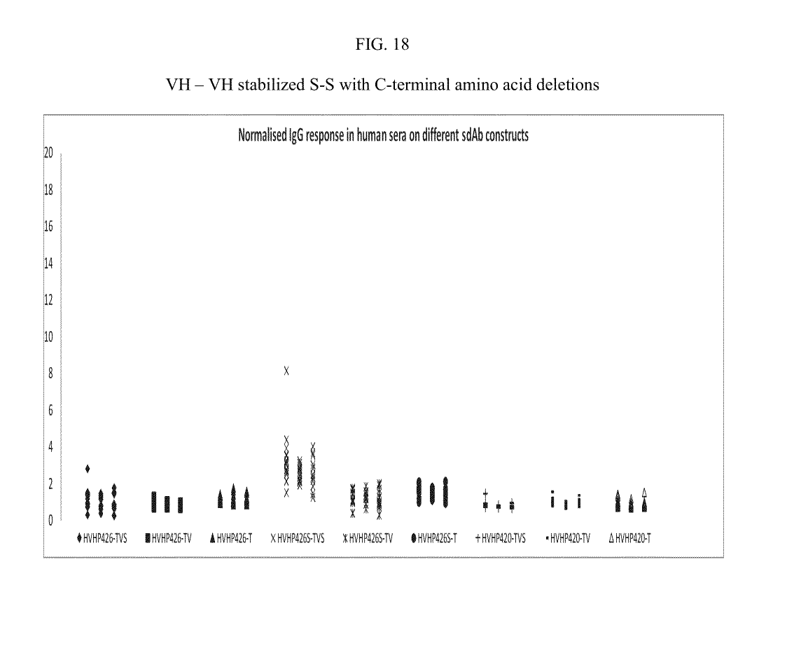

FIG. 19 shows the response in human sera with VH-VH stabilized S-S with single domain scaffolds HVHM81, and HVHP421S and HVHP430S with C-terminal amino acid deletions;

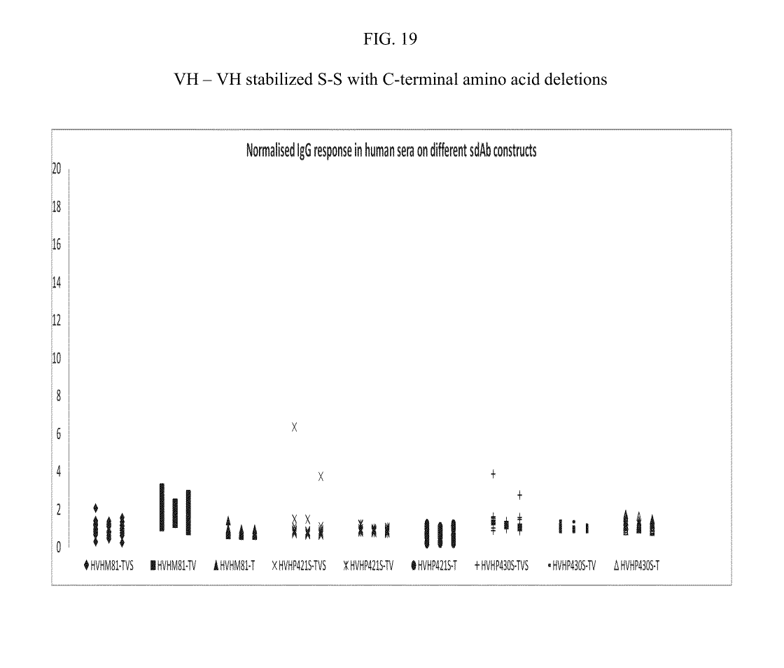

### DETAILED DESCRIPTION

In order to provide a clear understanding of the specification and claims, the following definitions are conveniently provided below.

The term “Antigen Binding Domain” or “ABD” as used herein refers to protein or a fragment of a protein with an exposed C-terminal that binds to an antigen. Examples of ABD include, but are not limited to, an antibody, a single domain antibody with an exposed C-terminus, an antibody variable region with an exposed C-terminus (e.g., V_{H} or V_{L}), a single chain antibody fragment (scFv) with an exposed C-terminus, a single chain diabody with an exposed C-terminus, a Fab fragment with at least one exposed C-terminus, a F(ab)₂ fragment with at least one exposed C-terminus, a bivalent fragment comprising two Fab fragments linked by a disulfide bridge at the hinge region, with at least one exposed C-terminus, a Fv fragment consisting of the V_{L} and V_{H} domains of a single arm of an antibody with at least one exposed C-terminus, and a dAb fragment with at least one exposed C-terminus. ABDs include, but are not limited to, single chain antibody, a nanobody, a multidomain antibody comprising fusions of IgGs or HSA with other ABD's such as a single chain Fv's, nanobodies or other small ABD's, a bispecific antibody comprising a single chain, a scFv, a sdAb, an Fab, a diabody, a scFab or any other ABD or Fc-fusion protein that will expose a normally not exposed N- or C-terminal sequence.

The term “C-terminal modification” as used herein refers to an ABD (e.g., a VH single domain antibody) with a modification at its exposed C-terminus that alters the structure of the C-terminus such that a pre-existing antibody no longer interacts with the modified C-terminus of the ABD (e.g., a VH single domain antibody). The alteration can be the addition of at least one amino acid residue to the C-terminus of the ABD (e.g., a VH single domain antibody), or the deletion of at least one amino acid residue from the C-terminus of the ABD (e.g., a VH single domain antibody). The addition of least one amino acid residue to the C-terminus of the ABD serves to mask or cap the exposed C-terminus such that pre-existing antibodies no longer interact with the masked or capped C-terminus by masking the neoepitope such that it is no longer available for interaction with the pre-existing antibodies. The deletion of at least one amino acid residue from the C-terminus of the ABD serves to modify the C-terminus by changing the neoepitope available for interaction by the pre-existing antibodies. The term “C-terminal modification” as it pertains to deletion of at least one amino acid residue, expressly relates to the deletion of at least one amino acid from an unmodified V_{H} framework, and specifically excludes any amino acids from the constant region of an ABD.

The term “N-terminal modification” as used herein refers to an ABD (e.g., single domain antibody) with a modification at its exposed N-terminus that alters the structure of the N-terminus such that a pre-existing antibody no longer interacts with the modified N-terminus of the ABD (e.g., single domain antibody). The alteration can be the addition of at least one amino acid residue to the N-terminus of the ABD (e.g., single domain antibody), or the deletion of at least one amino acid residue from the N-terminus of the ABD (e.g., single domain antibody). The addition of least one amino acid residue to the N-terminus of the ABD serves to mask or cap the exposed N-terminus such that pre-existing antibodies no longer interact with the masked or capped N-terminus by masking the neoepitope such that it is no longer available for interaction with the pre-existing antibodies. The deletion of at least one amino acid residue from the N-terminus of the ABD serves to modify the N-terminus by changing the neoepitope available for interaction by the pre-existing antibodies.

The term “C+N terminal modification” as used herein refers to an ABD (e.g., single domain antibody) with a modification at its exposed C+N termini that alters the structure of the C+N termini such that a pre-existing antibody no longer interacts with the modified C+N termini of the ABD (e.g., single domain antibody). The alteration can be the addition of at least one amino acid residue to both the C+N termini of the ABD (e.g., single domain antibody), or the deletion of at least one amino acid residue from both the C+N termini of the ABD (e.g., single domain antibody). The addition of least one amino acid residue to both the C+N termini of the ABD serves to mask or cap the exposed C+N termini such that pre-existing antibodies no longer interact with the masked or capped C+N termini by masking the neoepitope such that it is no longer available for interaction with the pre-existing antibodies. The deletion of at least one amino acid residue from both the C+N termini of the ABD serves to modify the C+N termini by changing the neoepitope available for interaction by the pre-existing antibodies. Also, different modifications can be made to each termini of the ABD. For example, the C-terminus of the ABD can be modified to add least one amino acid residue, while the N-terminus of the ABD can be modified to delete least one amino acid residue. Alternatively, the N-terminus of the ABD can be modified to add least one amino acid residue, while the C-terminus of the ABD can be modified to delete least one amino acid residue.

The term “immunogenicity” as used herein refers to the immunogenicity resulting from pre-existing antibodies that have existed prior to the administration of the ABD (e.g., single domain antibody). The immunogenicity resulting from pre-existing antibodies reduces the therapeutic effect of an ABD (e.g., single domain antibody). The extent of such immunogenicity can be determined by an ELISA assay and can be expressed as the percentage of human sera that contain measurable amounts of pre-existing antibodies. A reduction of immunogenicity between an ABD (e.g., single domain antibody) and a corresponding ABD (e.g., single domain antibody) with a modification, such as a C-terminal modification can be measured by comparing the percentage of serum samples containing pre-existing antibodies against the ABD (e.g., single domain antibody) with a C-terminal modification with the percentage of serum samples containing pre-existing antibodies against the original ABD (e.g., single domain antibody). A lower number or percentage of positive serum samples for the ABD (e.g., single domain antibody) with a C-terminal modification indicates a reduction of immunogenicity for the ABD (e.g., single domain antibody) with a C-terminal modification. A more sensitive measurement, which can be applied on the basis of a single serum sample, employs a competition ELISA setup. In such competition ELISA the ABD (e.g., single domain antibody) with a C-terminal modification competes with the original ABD (e.g., single domain antibody) for binding of pre-existing antibodies in the test serum. The lower the ability of the ABD (e.g., single domain antibody) with a C-terminal modification to compete with the original ABD (e.g., single domain antibody), the more successful the immunogenicity was reduced.

The term “single domain antibody” or “sdAb” as used herein refers to a type of single chain antibody comprising a variable region (V_{HH}) of a heavy chain of a human antibody. SdAbs are antibody fragments consisting of a single monomeric variable antibody domain. They are derived, for example, from heavy chain antibodies derived from humans, which consist only of two antibody heavy chains, with no light chain. With a molecular weight of only 12-15 kDa, sdAbs are much smaller than monoclonal antibodies (mAbs), e.g., IgG antibodies (150-160 kDa), which have two heavy protein chains and two light chains.

SdAbs may be derived from any species including, but not limited to mouse, human, camel, llama, goat, rabbit, bovine. The sdAb can be modified versions of a naturally occurring immunoglobulin known as heavy chain antibody devoid of light chains. Such immunoglobulins are disclosed in WO2006/099747; WO2009/079793; and WO2012/100343. For clarity reasons, the variable domain derived from a heavy chain antibody naturally devoid of light chain is known herein as a V_{HH} or sdAb to distinguish it from the conventional V_{H} of four chain immunoglobulins.

The term “pre-existing antibody” as used herein, refers to interfering antibodies in the serum or sera of a subject that are not induced as a result of administrating the ABD (e.g., single domain antibody), but rather are present in the subject prior to administrating the ABD (e.g., single domain antibody). Pre-existing antibodies may arise from incidental or occupational exposure to foreign proteins. These pre-existing antibodies interact with the exposed C-terminus of the ABD (e.g., single domain antibody), and reduce the therapeutic effect of the ABD (e.g., single domain antibody).

Various aspects of the disclosure are described in further detail in the following sections and subsections.

### Antigen Binding Domains

In one aspect, the disclosure pertains to an isolated antigen binding domain (ABD) comprising a C-terminal modification, wherein the C-terminal modification comprises the addition or deletion of at least one amino acid residue such that the addition or deletion of at least one amino acid residue to the ABD eliminates the interaction of a pre-existing antibody with the ABD without interfering with the binding of the ABD with its target. In another aspect, the invention pertains to an antigen binding domain (ABD) comprising a C-terminal modification, wherein the C-terminal modification comprises the deletion of at least one amino acid residue such that the deletion of at least one amino acid residue from the ABD eliminates the interaction of a pre-existing antibody with the ABD without interfering with the binding of the ABD with its target. In another aspect, the invention pertains to an antigen binding domain (ABD) comprising a C-terminal modification, wherein the C-terminal modification comprises the deletion of at least one amino acid residue to at least three amino acid residues such that the deletion of at least one amino acid residue from the ABD eliminates the interaction of a pre-existing antibody with the ABD without interfering with the binding of the ABD with its target. In another aspect, the disclosure pertains to an isolated antigen binding domain (ABD) comprising an N-terminal modification, wherein the N-terminal modification comprises the addition or deletion of at least one amino acid residue such that the addition or deletion of at least one amino acid residue to the ABD eliminates the interaction of a pre-existing antibody with the ABD without interfering with the binding of the ABD with its target. In another aspect, the invention pertains to an isolated antigen binding domain (ABD) comprising a C+N terminal modification, wherein the C+N terminal modification comprises the addition or deletion of at least one amino acid residue such that the addition or deletion of at least one amino acid residue to the ABD eliminates the interaction of a pre-existing antibody with the ABD without interfering with the binding of the ABD with its target.

Examples of ABD include, but are not limited to, an antibody, a single domain antibody with an exposed C-terminus, N-terminus, or C+N termini; an antibody variable region with an exposed C-terminus, N-terminus, or C+N termini; (e.g., V_{H} or V_{L}), a single chain antibody fragment (scFv) with an exposed C-terminus, N-terminus, or C+N termini; a single chain diabody with an exposed C-terminus, N-terminus, or C+N termini; a Fab fragment with at least one exposed C-terminus, N-terminus, or C+N termini; a F(ab)₂ fragment with at least one exposed C-terminus, N-terminus, or C+N termini; a bivalent fragment comprising two Fab fragments linked by a disulfide bridge at the hinge region, with at least one exposed C-terminus N-terminus, or C+N termini, a Fv fragment consisting of the V_{L} and V_{H} domains of a single arm of an antibody with at least one exposed C-terminus, N-terminus, or C+N termini; and a dAb fragment with at least one exposed C-terminus N-terminus, or C+N termini. ABDs include, but are not limited to, single chain antibody, a nanobody, a multidomain antibody comprising fusions of IgGs or HSA with other ABD's such as a single chain Fv's, nanobodies or other small ABD's, a bispecific antibody comprising a single chain, a scFv, a sdAb, an Fab, a diabody, a scFab or any other ABD or Fc-fusion protein that will expose a normally not exposed N- or C-terminal sequence.

The ABD has complementarity determining regions (CDR) along with up to four framework regions (FR) form the antigen-binding site. The CDR of the V_{H} or V_{L} variable domain are referred to herein as CDR1, CDR2, and CDR3. The FRs of the V_{H} or V_{L} variable domain are referred to herein as FR1, FR2, FR3 and FR4. The FR provide structural integrity to the variable domain and ensure retention of the immunoglobulin fold. Various schemes exist for identification of the complementarity-determining regions, the two most common being those of Kabat et al. (1991) define the “complementarity-determining regions” (CDR) based on sequence variability at the antigen-binding regions of the V_{H} and/or V_{L} domains (See Kabat et al. (1991) Sequences of Proteins of Immunological Interest. US Department of Health and Human Services, US Public Health Service, Bethesda, Md.). The majority of the sequence variability in variable domains (V_{H} or V_{L}) occurs in the CDR/loops; the regions outside the CDR/loops are referred to as the framework regions (FR). The FR and CDR regions of ABD may also be determined using the IMGT international database (See e.g., www.imgt.org; Lefranc, et al., (1999) Nucleic Acids Research, 27, 209-212 (1999); Ruiz, et al., (2000) Nucleic Acids Research, 28, 219-221; Lefranc, (2001) Nucleic Acids Research, 29, 207-209; Lefranc, (2003) Nucleic Acids Res., 31, 307-310; Lefranc, et al., (2004) In Silico Biol., 5, 0006 [Epub], 5, 45-60 (2005); Lefranc, et al., (2005) Nucleic Acids Res., 33, D593-D597).

Human V_{H} or V_{L} domains may be obtained from human Ig heavy or light chain sequences (Holliger, and Hudson, (2005) Nat. Biotechnol. 23, 1126-1136; Holt et al., (2003) Trends Biotechnol. 21, 484-490; Jespers et al, (2004) Nat. Biotechnol. 22, 1161-1165; To et al, (2005) J. Biol. Chem. 280, 41395-41403). Similar techniques are known in the art for obtaining V_{H} or V_{L} domains from non-human species. Furthermore, V_{H} and V_{L} domains include recombinantly produced V_{H} or V_{L}, as well as those V_{H} or V_{L} generated through further modification of such V_{H} or V_{L} by affinity maturation, stabilization, solubilization or other methods of antibody engineering. Also encompassed are homologues, derivatives, or variants that retain or improve the stability and non-aggregation characteristics of the V_{H} or V_{L}.

For the purpose of illustration only, the following section describes using the disclosure in the context of single domain antibodies, however it is to be understood that the disclosure is applicable to a number of antibodies, antibody fragments, or combinations thereof that have at least one exposed C-terminus, N-terminus, or C+N termini that elicits an immune response to pre-existing antibodies, wherein modification of the exposed C-terminus, N-terminus, or C+N termini, by addition or deletion of amino acids at the C-terminus, N-terminus, or C+N termini, eliminates the response. Likewise, the disclosure also applies to a antibody-alternative scaffolds (e.g., fibronectin) that have at least one exposed C-terminus, N-terminus, or C+N termini that elicits an immune response to pre-existing antibodies, wherein modification of the exposed C-terminus N-terminus, or C+N termini, by addition or deletion of amino acids at the C-terminus, N-terminus, or C+N termini, eliminates the response.

### Human Single Domain Antibodies

The invention is based on the surprising discovery that pre-existing antibodies in human sera bind to human single domain antibodies and produce a pre-existing immune response. As the single domain antibodies are of human origin and not derived or modified versions of single domain antibodies from other species, Applicants were surprised to discover that pre-existing antibodies present in sera from human volunteers bound to several single domain antibodies disclosed herein, to produce a pre-existing immune response. Even more surprising and unexpected, was the finding that modifications (addition or deletion of a single amino acid residue) at the C-terminal region of a human V_{H} single domain antibody, eliminates the pre-existing immune response. Based on the data disclosed herein, there is no evidence however, that there is a pre-existing immune response to the C-terminus of a human V_{L} single domain antibody, as defined by the IMGT database convention (See e.g., www.imgt.org; Lefranc, et al., (1999), Supra; Ruiz, et al., (2000) Supra; Lefranc, (2001) Supra; Lefranc, (2003) Supra; Lefranc, et al., (2004) Supra; Lefranc, et al., (2005) Supra).

Although single domain antibodies derived from camelid heavy chain (V_{HH}) demonstrate some pre-existing immune response, even with humanizing mutations (See e.g., WO2012/175741, it is not surprising that there may be some pre-existing antibodies that may react with the camelid heavy chain. For the camelid heavy chain, modifications to the C-terminus that added amino acid residues to the C-terminus reduced the pre-existing immune response. Such amino acid additions were also shown to reduce a pre-existing immune response in WO2013/024059.

However, to date, there is little evidence that pre-existing antibodies bind to a human single domain antibody, and that this binding can be eliminated by the addition or deletion of at least one amino acid residue from the C-terminal region of an unmodified human single domain antibody scaffold as disclosed herein. Applicants disclose for the first time, that a deletion of a single amino acid from the C-terminal region of the unmodified V_{H} human single domain antibody scaffold is enough to eliminate the pre-existing immune response. Even more surprising is that the elimination of the pre-existing immune response was seen only with V_{H} single domain antibody scaffolds, but there was no evidence of it with the V_{L} single domain antibody scaffolds.

Accordingly, in another aspect, the invention pertains to an isolated V_{H} single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target. In one embodiment, the single domain antibody is a human V_{H} single domain antibody, or derived from a human origin.

In one embodiment, the single domain antibody may be derived from a V_{H} region, a V_{HH} region or a V_{L} region. The single domain antibody V_{H} and V_{L} scaffolds are generated as described in the Examples section, using the PhoA leader sequence, MKQSTIALALLPLLFTPVTKA (SEQ ID NO: 1), which is used to purify the protein. However, once expressed and purified, the PhoA leader sequence is removed, retaining the single domain antibody V_{H} and V_{L} sequences shown below (SEQ ID NOs: 2-13).

In one embodiment, the human single domain antibody comprises heavy or light chain sequences disclosed in WO2006/099747 and WO2009/079793 and WO2012/100343, incorporated herein by reference in their entirety. In one embodiment, the human single domain antibody comprises heavy or light chain sequences with a disulfide bonds within the framework region as discussed in WO2012/100343. To date, no detectable amounts of free circulating human single domain antibodies have been identified in the human system.

In one embodiment, the human single domain antibody comprises heavy or light chain sequences selected from the group consisting of:

Light Chain Domain
HVLP335
(SEQ ID NO: 2)
EIVMTQSPATLSLSPGERATLSCRASQSVSSSSLAWYQQKPGQAPRLLIYGTSNRATGIP
DRFSGSGSGTHFTLTINRLEPGDFAVYYCQQYGSSPRTFGQGTKVEIK
HVLP3103S
(SEQ ID NO: 3)
ETTLTQSPGTLSLSPGERATLSCRASQSVRNNLAWYQQRPGQAPRLLCYGASTRATGIP
ARFSCSGSGTDFTLTISSLQVEDVAVYYCQQYYTTPK-TFGQGTKVEIK
HVLP325
(SEQ ID NO: 4)
EIVLTQSPTTLSLSPGERATLSCRASQSVGR-
YLAWYQQRPGQAPRLLVFDTSNRAPGVPARFSGRGSGTLFTLTISSLEPEDSAVYFCQQ
RSSGL--TFGGGTKVTVL
HVLP325S
(SEQ ID NO: 5)
EIVLTQSPTTLSLSPGERATLSCRASQSVGR-
YLAWYQQRPGQAPRLLCFDTSNRAPGVPARFSCRGSGTLFTLTISSLEPEDSAVYFCQQ
RSSGL--TFGGGTKVTVL
HVLP351
(SEQ ID NO: 6)
EIVMTQSPVTLSLSPGERATLSCRASQSVGT-
SLAWYQQKPGQAPRLLIYDASNRATGISARFSGSGSGTDFTLTISSLEPEDFAVYYCQQ
RYNWPR-TFGGGTKVTVL
HVLP351S
(SEQ ID NO: 7)
EIVMTQSPVTLSLSPGERATLSCRASQSVGT-
SLAWYQQKPGQAPRLLCYDASNRATGISARFSCSGSGTDFTLTISSLEPEDFAVYYCQQ
RYNWPR-TFGGGTKVTVL
Heavy Chain Domain
HVHP426
(SEQ ID NO: 8)
QVQLVQSGGGVVQPGRSLRLSCAASGFIVDGYAMHWVRQAPGQGLEWVSVTNNGGS
TSYADSVKG---RFTISRDNSKNTVYLQMNSLRAEDTAVYYCARQSITGPTGAFDI----
WGQGTMVTVSS
HVHP426S
(SEQ ID NO: 9)
QVQLVQSGGGVVQPGRSLRLSCAASGFIVDGYAMHWVRQAPGQGLEWVCVTNNGGS
TSYADSVKG---RFTCSRDNSKNTVYLQMNSLRAEDTAVYYCARQSITGPTGAFDI----
WGQGTMVTVSS
HVHP420
(SEQ ID NO: 10)
QVQLVESGGGLVKPGGSLRLSCAASGFTFSNAWMTWVRQAPGKGLEWVGRIKTKTD
GGTTDYAAPVKGRFTISRDDSKNTLYLQMNSLKTEDTAVYYCTTDRDHSSGS--------
WGQGTLVTVSS
HVHM81
(SEQ ID NO: 11)
EVQLVQSGGGLVQPGRSLRLSCAASGFTFDDYAMHWVRQAPGKGLEWVSGISGSGAS
TYYADSVKG--RFTISRDNSKNTLYLQMNSLRAGDTALYYCARQSITGPTGAFDV----
WGQGTMVTVSS
HVHP421S
(SEQ ID NO: 12)
QLQLQESGGGVVQPGRSLRLSCAASGFTFSSYAMSWVRQAPGKGLEWVCAISGSGGS
TYYADSVKG-
RFTCSRDNSKNTLYLQMNSLRAEDTAVYYCAKDGKGGSSGYDHPDYWGQGTLVTVS
S
HVHP430S
(SEQ ID NO: 13)
QVQLVESGGGLIKPGGSLRLSCAASGFTFSNYAMSWVRQAPGKGLEWVCAISSSGGST
YYADSVKG--
RFTCSRDNSKNTVYLQMNSLRAEDTAVYYCVREEYRCSGTSCPGAFDIWGQGTMVTV
SS

In one embodiment the single domain antibody comprises one or more non-canonical Cys residues described in WO2012/100343, incorporated herein by reference in its entirety, as well as any native (canonical) disulfide bonds. In one embodiment, at least two non-canonical Cys residues are introduced into the framework regions of the single domain antibody. In one embodiment, the two Cys residues replace residues in the FR2 and FR3 of an antibody variable region. In one embodiment, a Cys residue is introduced at a position selected from residues 47 to 49 of a V_{H} FR2 regions and a Cys residue at a position selected from residues 69 to 71 of a V_{H} FR3 regions of a V_{H} sdAb domain. In one embodiment, a Cys residue is introduced at a position selected from residues 46 to 49 of a V_{L} FR2 regions and a Cys residue at a position selected from residues 62 to 66 of a V_{L} FR3 regions of a V_{L} sdAb domain. In one embodiment, at least one non-canonical Cys residue is introduced at position 49 and at least one non-canonical Cys residue at position 69 of a V_{H} domain. In one embodiment, at least one non-canonical Cys residue is introduced at position 48 and at least one non-canonical Cys residue at position 64 of a V_{L} domain.

In one embodiment the single domain antibody is “humaneered” (also alternatively termed “humanized”), i.e., a sdAb that originated from a species other than human that has had immunogenic or potentially immunogenic amino acid residues replaced with amino acids that are less immunogenic or not immunogenic in the context of a sdAb administered to a human subject. Any method known in the art for creating humaneered antibodies are contemplated in the disclosure, including but not limited to humaneering technology of Kalobios. Note that when a single domain antibody is humaneered, an immunogenic amino acid residue may be replaced by any other less immunogenic amino acid residue regardless of whether or not this constitutes a conserved amino acid change.

### Pre-Existing Antibodies

Pre-existing antibodies can be formed early in life and are subject dependent. With the development of monoclonal antibodies and antibody fragments as novel therapeutics, the existence of pre-existing antibodies restricts and complicates the usage of therapeutic antibodies.

The mechanisms for interaction of pre-existing antibodies remains poorly understood, despite their detrimental effects in the clinical setting highlighting its serious consequences. The potential underlying mechanisms of interference from pre-existing antibodies appears to be an insidious, variable, and unpredictable problem irrespective of the immunoassay used to detect the pre-existing antibodies, the assay design or format. Pre-existing antibodies are generally polyclonal. The binding affinity of an antigen to a monoclonal antibody (i.e., the association constant, or Ka, which is a ratio of two rates, the association and dissociation rates) tends to be uniform, whereas in an immunologic reaction involving polyclonal antibodies (reagent or interfering antibodies), the Ka or avidity is a mean value of binding constants of each of the antibody populations. Immunologic reactions involving polyclonal antibodies are therefore more variable and complex. Furthermore, pre-existing antibodies may mimic the antigen itself, mimic the reagent antibodies, or mimic both, e.g., in idiotype network immune response (Pan et al. (1995) Exp Biol Med J; 9:43-49; Weir et al. Immunology 1997:69 Churchill Livingstone Edinburgh; Fields et al. (1995) Nature; 374:739-742). Some pre-existing antibodies do not recognize either the antigen or the reagent antibodies as entities for interaction, but they may recognize the “antigen-antibody bound complex” (i.e., metatope) and bind to it (Voss E W (1993) Mol Immunol; 30:949-951), thus altering the immunoassay kinetics. The normal binding reaction may therefore involve more than one binding site on the antigen and/or on the antibody. The presence of pre-existing antibodies could disrupt this binding reaction, and the magnitude of disruption would be dependent on factors such as the titer of pre-existing antibodies, their avidities and reaction times, and the location(s) of antibody binding site(s). The site(s) of binding of pre-existing antibodies on the capture antibodies could lead to blocking of the binding to the antigen (partially or completely), giving falsely low results. Alternatively, it could increase the binding with signaling antibodies by binding to a distal site on the capture antibody but reacting with the signaling antibody, giving a falsely higher result with the latter.

The variations in the nature of pre-existing antibodies in terms of class (IgG, IgM, or IgA), subclass (e.g., IgG1, -2, or -3), titer and affinities/avidities, and the multiplicity of epitopes/paratopes are only examples that highlight the complexity and unpredictability of binding reactions when potentially pre-existing antibodies and illustrates immunologic interactions that could give rise to numerous scenarios and permutations of interference.

The binding of a single domain antibody by pre-existing antibody can change the pharmacokinetic and pharmacodynamic behavior of the single domain antibody, create new complexes and functions; and changes the size of the complex which may also have consequences for tissue distribution. Therefore, this phenomenon represents a significant safety and efficacy risk which needs to be avoided.

Applicants believe that the pre-existing immune response is elicited by any protein that will expose a normally unexposed N- or C-terminal sequence. The disclosure seeks to eliminate the interference effects of pre-existing antibodies by modifying the C-terminus, the N-terminus, or both the C+N termini of an ABD such that the pre-existing antibodies no longer interact with the C-terminus, the N-terminus, or both the C+N termini of an ABD. There are a number of permutations that can be used to modify such a response. In one embodiment, the C-terminus is modified in which the addition of at least one amino acid residue to the C-terminus of the ABD. In another embodiment, the C-terminus is modified by the deletion of at least one amino acid residue from the C-terminus of the ABD. In one embodiment, the N-terminus is modified by the addition of at least one amino acid residue to the N-terminus of the ABD. In another embodiment, the N-terminus is modified by the deletion of at least one amino acid residue from the N-terminus of the ABD. In one embodiment, the C-terminus is modified by the addition of at least one amino acid residue to the C-terminus of the ABD, while the N-terminus is modified by the deletion of at least one amino acid residue from the N-terminus of the ABD. In one embodiment, the N-terminus is modified by the addition of at least one amino acid residue to the N-terminus of the ABD, while the C-terminus is modified by the deletion of at least one amino acid residue from the C-terminus of the ABD.

While not being bound to provide a theory, the modification to the C-terminus, the N-terminus, or both the C+N termini of the ABD may result in (i) eliminating the interaction of the pre-existing antibody by altering the three dimensional configuration of the the C-terminus, the N-terminus, or both the C+N termini of the ABD such that the pre-existing antibody no longer recognizes the ABD, (ii) alter the exposure of the C-terminal, N-terminal, or both the C+N termini of the ABD to the pre-existing antibody, (iii) alter the steric hindrance between the ABD and the pre-existing antibody, (iv) disrupt at least one conformational neoepitope in the C-terminus, N-terminus, or separate neoepitopes in both the C+N termini; and/or (v) shield at least one neoepitope in framework of the ABD.

By masking or capping the exposed C-terminus, N-terminus, or both the C+N termini of the ABD with the addition of at least one amino acid, the conformation of the termini may change; or the neoepitope may no longer be available for interaction with the pre-existing antibodies. Likewise, the deletion of at least one amino acid residue from the C-terminus, the N-terminus, or both the C+N termini of the ABD serves to modify the termini by changing the conformation at the termini or the neoepitope rendering them no longer available for interaction by the pre-existing antibodies.

In one embodiment, the disclosure seeks to eliminate the interference effects of pre-existing antibodies by modifying the C-terminus of an isolated single domain antibody such that the pre-existing antibodies no longer interact with the C-terminus of the single domain antibody. In one embodiment, the C-terminus is modified by the addition of at least one amino acid residue to the C-terminus of the single domain antibody. In another embodiment, the C-terminus is modified by the deletion of at least one amino acid residue from the C-terminus of the single domain antibody.

While not being bound to provide a theory, the modification to the C-terminus of the single domain antibody may result in (i) eliminating the interaction of the pre-existing antibody by altering the three dimensional configuration of the C-terminal single domain antibody such that the pre-existing antibody no longer recognizes the single domain antibody, (ii) alter the exposure of the C-terminal single domain antibody to the pre-existing antibody, (iii) alter the steric hindrance between the single domain antibody and the pre-existing antibody, (iv) disrupt at least one conformational neoepitope in the C-terminus, and/or (v) shield at least one neoepitope in framework of the single domain antibody.

In one embodiment, the single domain antibody comprises an exposed C-terminus peptide that comprises a neoepitope and the pre-existing antibodies specifically interact with the C-terminal neoepitope. The addition of at least one amino acid residue to the C-terminus of the single domain antibody, or the or deletion of at least one amino acid from the C-terminus of the single domain antibody, alters the structure of the neoepitope such that the pre-existing antibody no longer recognizes the neoepitope and no longer interacts with the C-terminus of the single domain antibody.

The data disclosed herein shows that the exposed C-terminus of the isolated single domain antibody is a preferred epitope for interaction of pre-existing antibodies. In one embodiment, the neoepitope at the exposed C-terminus of the single domain antibody that is recognized by pre-existing antibodies can be masked by addition of at least one amino acid residue to the C-terminus of the single domain antibody.

In one embodiment, the neoepitope at the C-terminus of the isolated single domain antibody is masked with the addition of at least one amino acid residue to the C-terminus of the single domain antibody. In another embodiment, the neoepitope at the C-terminus of the isolated single domain antibody is masked with the addition of at two one amino acid residues to the C-terminus of the single domain antibody. In another embodiment, the neoepitope at the C-terminus of the isolated single domain antibody is masked with the addition of at least three amino acid residues to the C-terminus of the single domain antibody. In another embodiment, the neoepitope at the C-terminus of the isolated single domain antibody is masked with the addition of at least four amino acid residues to the C-terminus of the single domain antibody. In one another embodiment, the neoepitope at the C-terminus of the isolated single domain antibody is masked with the addition of at least five amino acid residues to the C-terminus of the single domain antibody. In another embodiment, the neoepitope at the C-terminus of the isolated single domain antibody is masked with the addition of at least six amino acid residues to the C-terminus of the single domain antibody. In another embodiment, the neoepitope at the C-terminus of the isolated single domain antibody is masked with the addition of at least seven amino acid residues to the C-terminus of the single domain antibody. In another embodiment, the neoepitope at the C-terminus of the isolated single domain antibody is masked with the addition of at least eight amino acid residues to the C-terminus of the single domain antibody. In another embodiment, the neoepitope at the C-terminus of the isolated single domain antibody is masked with the addition of at least nine amino acid residues to the C-terminus of the single domain antibody. In another embodiment, the neoepitope at the C-terminus of the isolated single domain antibody is masked with the addition of at least ten amino acid residues to the C-terminus of the single domain antibody. In another embodiment, the neoepitope at the C-terminus of the isolated single domain antibody is masked with the addition of at least 1-10 amino acid residues to the C-terminus of the single domain antibody.

In one embodiment, the neoepitope at the C-terminus of the single domain antibody is altered with addition or deletion of the amino acid residues disclosed in Table 1.

TABLE 1
C-terminal modifications

| | Amino acid sequence |
|---|---|
| 1) | Wild type - no amino acid |
| | extension |
| 2) | Ala |
| 3) | Ala-Ala |
| 4) | Ala-Ser |
| 5) | Ala-Ser-Thr |
| 6) | Ala-Ser-Thr-Lys-Pro |
| | (SEQ ID NO: 14) |
| 7) | Gly-Gly-Gly-Gly-Ser |
| | (SEQ ID NO: 15) |
| 8) | Gly |
| 9) | Gly-Gly |
| 10) | Gly-Ser |
| 11) | Arg deletion |
| 12) | Arg-Thr deletion |
| 13) | Arg-Thr-Val deletion |
| 14) | Gly-Gln deletion |
| 15) | Gly-Gln-Pro deletion |

In one embodiment, the neoepitope at the C-terminus of the isolated single domain antibody is masked with the addition of Ala to the C-terminus of the single domain antibody. In one embodiment, the neoepitope at the C-terminus of the isolated single domain antibody is masked with the addition of Ala-Ala to the C-terminus of the single domain antibody. In one embodiment, the neoepitope at the C-terminus of the isolated single domain antibody is masked with the addition of Ala-Ser to the C-terminus of the single domain antibody. In one embodiment, the neoepitope at the C-terminus of the isolated single domain antibody is masked with the addition of Ala-Ser-Thr to the C-terminus of the single domain antibody. In one embodiment, the neoepitope at the C-terminus of the isolated single domain antibody is masked with the addition of Ala-Ser-Thr-Lys-Pro (SEQ ID NO: 14) to the C-terminus of the single domain antibody. In one embodiment, the neoepitope at the C-terminus of the isolated single domain antibody is masked with the addition of Gly-Gly-Gly-Gly-Ser (SEQ ID NO: 15) to the C-terminus of the single domain antibody. In one embodiment, the neoepitope at the C-terminus of the isolated single domain antibody is masked with the addition of Gly to the C-terminus of the single domain antibody. In one embodiment, the neoepitope at the C-terminus of the isolated single domain antibody is masked with the addition of Gly-Gly to the C-terminus of the single domain antibody. In one embodiment, the neoepitope at the C-terminus of the isolated single domain antibody is masked with the addition of Gly-Ser to the C-terminus of the single domain antibody.

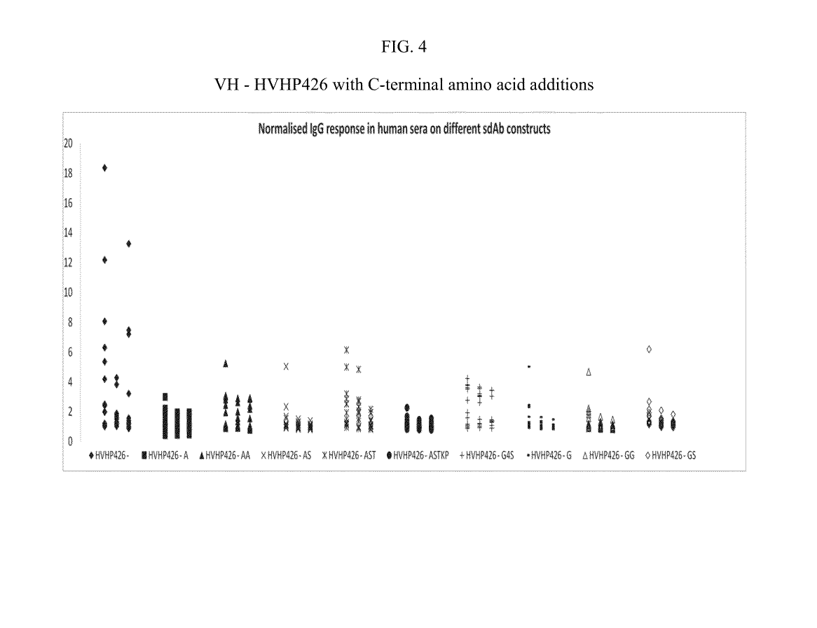

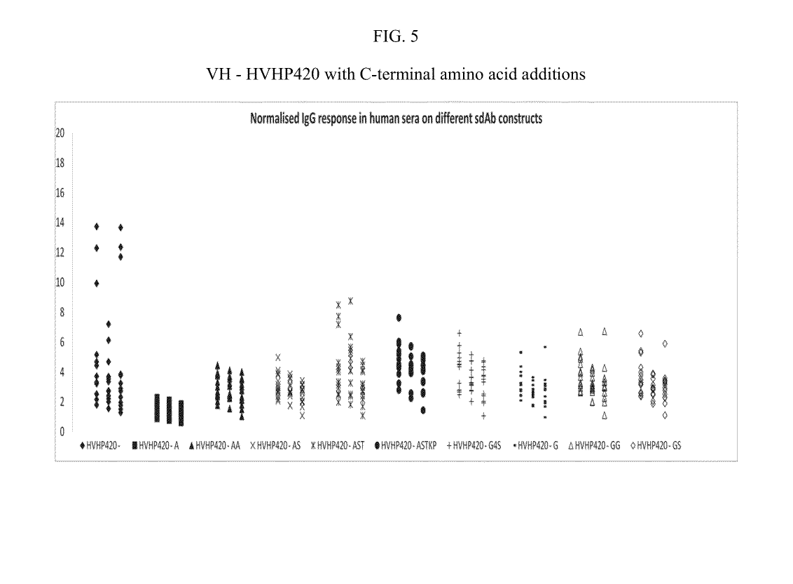

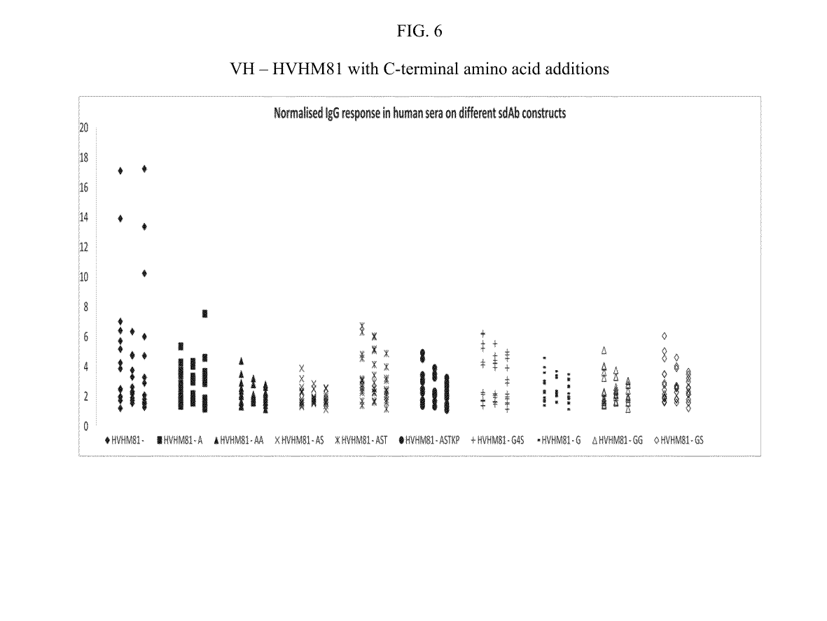

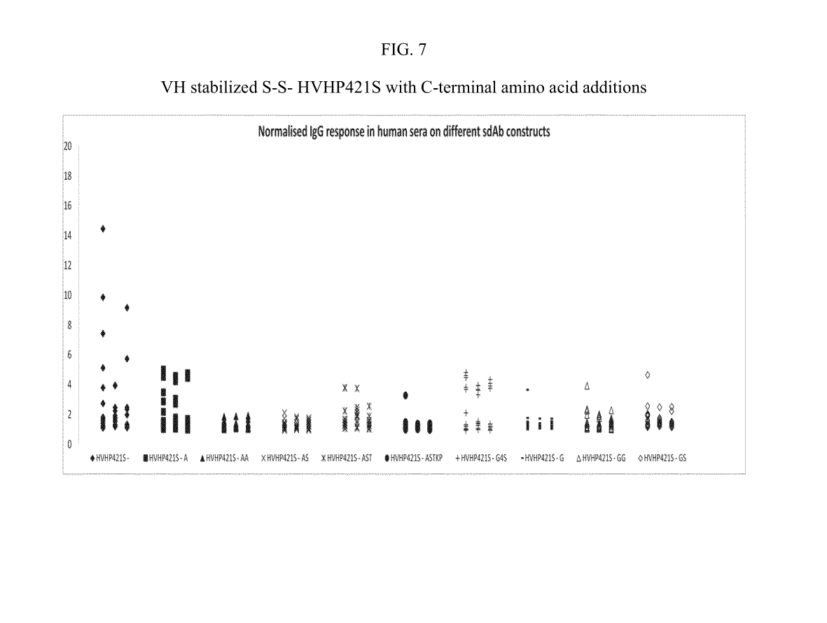

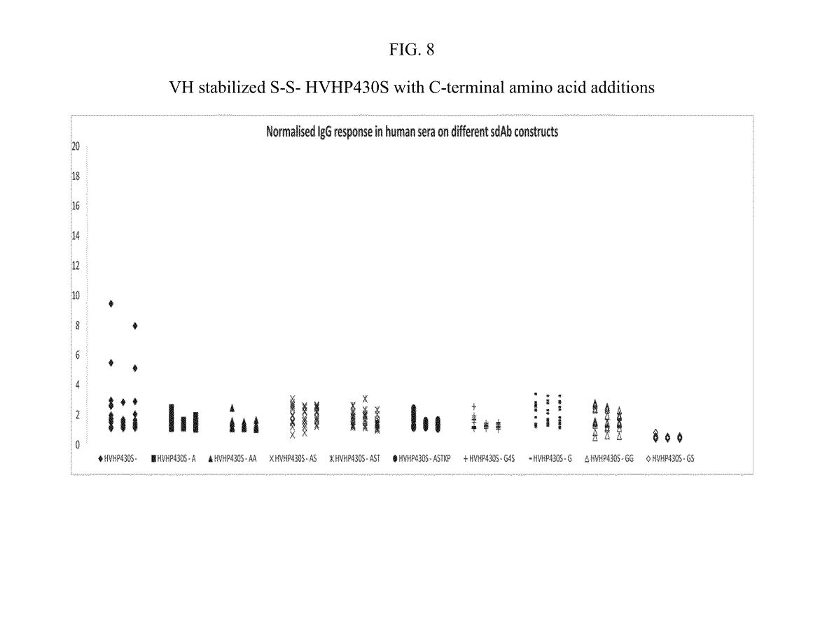

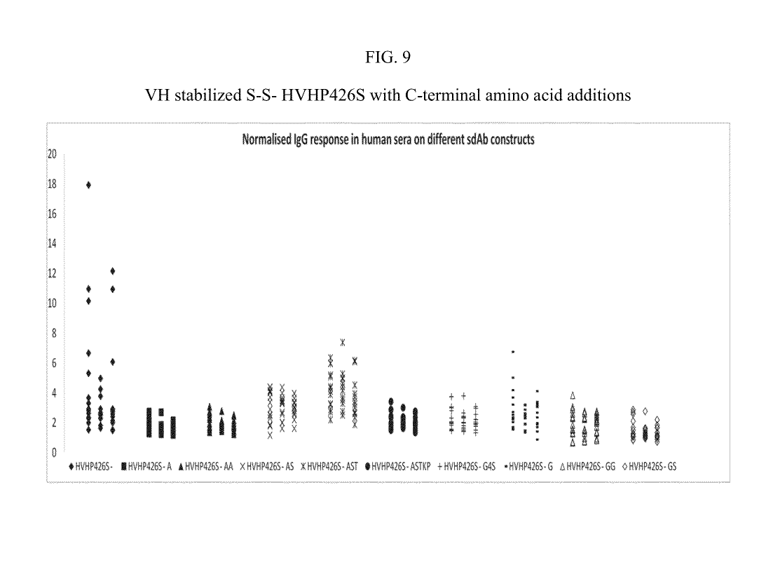

FIGS. 4-9 show the presence of pre-existing antibody response to a number of wild type V_{H} single domain scaffolds. These Figures clearly show that with each of the scaffolds, there is complete elimination, or a significant reduction of the pre-existing antibody response with the addition of one or more amino acid residues at the C-terminus of each V_{H} single domain scaffold. The data also shows there is a tendency for an increased response for the Ala-Ser-Thr amino acid addition compared with other additions. The data also shows a higher response for V_{H} versus stabilized V_{H} S-S scaffolds. Interestingly, no pre-existing antibody response was detected in the current assays with a number of wild type V_{L} single domain scaffolds.

In one embodiment, the neoepitope at the exposed C-terminus of the single domain antibody that is recognized by pre-existing antibodies can be eliminated by deletion of at least one amino acid residue from the C-terminus of the single domain antibody.

In one embodiment, the neoepitope at the exposed C-terminus of the single domain antibody that is recognized by the pre-existing antibody is eliminated by the deletion of at least one amino acid residue from the C-terminus of the single domain antibody. In another embodiment, the neoepitope at the exposed C-terminus of the single domain antibody that is recognized by the pre-existing antibody is eliminated by the deletion of at two one amino acid residues from the C-terminus of the single domain antibody. In another embodiment, the neoepitope at the exposed C-terminus of the single domain antibody that is recognized by the pre-existing antibody is eliminated by the deletion of at least three amino acid residues from the C-terminus of the single domain antibody. In another embodiment, the neoepitope at the exposed C-terminus of the single domain antibody that is recognized by the pre-existing antibody is eliminated by the deletion of at least four amino acid residues from the C-terminus of the single domain antibody. In one embodiment, the neoepitope at the exposed C-terminus of the single domain antibody that is recognized by the pre-existing antibody is eliminated by the deletion of at least five amino acid residues from the C-terminus of the single domain antibody. In another embodiment, the neoepitope at the exposed C-terminus of the single domain antibody that is recognized by the pre-existing antibody is eliminated by the deletion of at six amino acid residues from the C-terminus of the single domain antibody. In another embodiment, the neoepitope at the exposed C-terminus of the single domain antibody that is recognized by the pre-existing antibody is eliminated by the deletion of at least seven amino acid residues from the C-terminus of the single domain antibody. In another embodiment, the neoepitope at the exposed C-terminus of the single domain antibody that is recognized by the pre-existing antibody is eliminated by the deletion of at least eight amino acid residues from the C-terminus of the single domain antibody. In another embodiment, the neoepitope at the exposed C-terminus of the single domain antibody that is recognized by the pre-existing antibody is eliminated by the deletion of at nine amino acid residues of the C-terminus. In another embodiment, the neoepitope at the exposed C-terminus of the single domain antibody that is recognized by the pre-existing antibody is eliminated by the deletion of at least ten amino acid residues from the C-terminus of the single domain antibody. In another embodiment, the neoepitope at the exposed C-terminus of the single domain antibody that is recognized by the pre-existing antibody is eliminated by the deletion of at least 1-10 amino acid residues from the C-terminus of the single domain antibody.

In one embodiment, the neoepitope at the C-terminus of the isolated single domain antibody is eliminated by the deletion of Arg at the C-terminus of the single domain antibody. In one embodiment, the neoepitope at the C-terminus of the isolated single domain antibody is eliminated by the deletion of Arg-Thr at the C-terminus of the single domain antibody. In one embodiment, the neoepitope at the C-terminus of the isolated single domain antibody is eliminated by the deletion of Arg-Thr-Val at the C-terminus of the single domain antibody. In one embodiment, the neoepitope at the C-terminus of the isolated single domain antibody is eliminated by the deletion of Gly-Gln at the C-terminus of the single domain antibody. In one embodiment, the neoepitope at the C-terminus of the isolated single domain antibody is eliminated by the deletion of Gly-Gln-Pro at the C-terminus of the single domain antibody.

In one embodiment, the isolated single domain antibody comprises a V_{H} comprising SEQ ID NO: 8 and the C-terminal modification comprises a deletion of one amino acid to three amino acids. In one embodiment, the isolated single domain antibody comprises a V_{H} comprising SEQ ID NO: 9 and the C-terminal modification comprises a deletion of one amino acid to three amino acids. In one embodiment, the isolated single domain antibody comprises a V_{H} comprising SEQ ID NO: 10 and the C-terminal modification comprises a deletion of one amino acid to three amino acids. In one embodiment, the isolated single domain antibody comprises a V_{H} comprising SEQ ID NO: 11 and the C-terminal modification comprises a deletion of one amino acid to three amino acids. In one embodiment, the isolated single domain antibody comprises a V_{H} comprising SEQ ID NO: 12 and the C-terminal modification comprises a deletion of one amino acid to three amino acids. In one embodiment, the isolated single domain antibody comprises a V_{H} comprising SEQ ID NO: 13 and the C-terminal modification comprises a deletion of one amino acid to three amino acids.

### Homologous Single Domain Antibodies

In another aspect, the disclosure pertains to isolated single domain antibodies with an exposed C-terminal comprising V_{H} or V_{L} that are homologous to an amino acid sequence selected from the group consisting of SEQ ID NO: 8, SEQ ID NO: 9, SEQ ID NO: 10, SEQ ID NO: 11, SEQ ID NO: 12, and SEQ ID NO: 13, and SEQ ID NO: 2, SEQ ID NO: 3, SEQ ID NO: 4, SEQ ID NO: 5, SEQ ID NO: 6, and SEQ ID NO: 7, wherein the C-terminus of the single domain antibody has been modified by either the addition or deletion of at least one amino acid residue such that the addition or deletion of at least one amino acid residue eliminates the interaction of a pre-existing antibody with the single domain antibody without interfering with the binding of the single domain antibody with its target.

In one embodiment, the invention pertains to V_{H} single domain antibody with an exposed C-terminal that is homologous to an amino acid sequence selected from the group consisting of SEQ ID NO: 8, SEQ ID NO: 9, SEQ ID NO: 10, SEQ ID NO: 11, SEQ ID NO: 12, and SEQ ID NO: 13, wherein the C-terminus of the single domain antibody has been modified by deletion of at least one amino acid residue such that the deletion of at least one amino acid residue eliminates the interaction of a pre-existing antibody with the single domain antibody without interfering with the binding of the single domain antibody with its target.

In one embodiment, the invention pertains to V_{L} single domain antibody with an exposed C-terminal that is homologous to an amino acid sequence selected from the group consisting of SEQ ID NO: 2, SEQ ID NO: 3, SEQ ID NO: 4, SEQ ID NO: 5, SEQ ID NO: 6, and SEQ ID NO: 7, wherein the C-terminus of the single domain antibody has been modified by deletion of at least one amino acid residue such that the deletion of at least one amino acid residue eliminates the interaction of a pre-existing antibody with the single domain antibody without interfering with the binding of the single domain antibody with its target.

In one embodiment, the disclosure provides an isolated V_{H} comprising an amino acid sequence that is at least 50%, 60%, 70%, 80%, 90%, 95%, 96%, 97%, 98% or 99% identical to an amino acid sequence selected from the group consisting of SEQ ID NO: 8, SEQ ID NO: 9, SEQ ID NO: 10, SEQ ID NO: 11, SEQ ID NO: 12, and SEQ ID NO: 13. In another embodiment, the disclosure provides an isolated V_{L} comprising an amino acid sequence that is at least 50%, 60%, 70%, 80%, 90%, 95%, 96%, 97%, 98% or 99% identical to an amino acid sequence selected from the group consisting of SEQ ID NO: 2, SEQ ID NO: 3, SEQ ID NO: 4, SEQ ID NO: 5, SEQ ID NO: 6, and SEQ ID NO: 7. Also included within the scope of the disclosure are V_{H} and V_{L} parental nucleotide sequences optimized for expression in a mammalian cell. Also within the scope of the disclosure are amino acids or nucleic acids that have been mutated, yet have at least 60%, 70%, 80%, 90%, 95%, 98%, or 99% percent identity to the sequences described above. In some embodiments, they include mutant amino acid sequences wherein no more than 1, 2, 3, 4 or 5 amino acids have been mutated by amino acid deletion, insertion or substitution in the V_{H} or V_{L} when compared with the sequences described above.

Also within the scope of the disclosure are isolated single domain antibodies with an exposed C-terminal with conservative modifications, wherein the C-terminal of the single domain antibody has been modified by either the addition or deletion of at least one amino acid residue such that the addition or deletion of at least one amino acid residue to the single domain antibody eliminates the interaction of a pre-existing antibody with the single domain antibody without interfering with the binding of the single domain antibody with its target.

For polypeptide sequences, “conservative sequence modifications” include individual substitutions, deletions or additions to a polypeptide sequence which results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The phrases “percent identical” or “percent identity,” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### Engineered and Modified Single Domain Antibodies

In another aspect, the isolated single domain antibody with an exposed C-terminal that has been modified to eliminate interaction with pre-existing antibodies can be further engineered using, for example, one or more of the V_{H} and/or V_{L} sequences of a single domain antibody as starting material to engineer a modified single domain antibody which may have altered properties from the starting single domain antibody. The isolated single domain antibody with the exposed C-terminal can be engineered by modifying one or more residues within one or both V_{H} and/or V_{L} sequences, for example within one or more CDR regions and/or within one or more framework regions.

One type of variable region engineering that can be performed is CDR grafting. Single domain antibodies interact with target antigens predominantly through amino acid residues that are located in the CDRs. For this reason, the amino acid sequences within CDRs are more diverse between individual single domain antibodies than sequences outside of CDRs. Because CDR sequences are responsible for most antibody-antigen interactions, it is possible to express recombinant single chain antibodies that mimic the properties of specific wild type single chain antibodies by constructing expression vectors that include CDR sequences from the wild type single chain antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.).

In another embodiment, the isolated single domain antibodies can be modified in their framework region, for example by the addition of one or more Cys residues. For example, single domain antibodies may comprise a polypeptide sequence comprising at least two non-canonical Cys residues introduced into the framework regions FR2 and FR3 of an antibody variable region. In one embodiment, the polypeptide comprises a Cys residue at a position selected from residues 47 to 49 of a V_{H} FR2 regions and a Cys residue at a position selected from residues 69 to 71 of a V_{H} FR3 regions of a V_{H} sdAb domain. In one embodiment, the polypeptide comprises a Cys residue at a position selected from residues 46 to 49 of a V_{L} FR2 regions and a Cys residue at a position selected from residues 62 to 66 of a V_{L} FR3 regions of a V_{L} sdAb domain, as discussed Supra (See WO2012/100343, incorporated herein by reference).

### Bispecific and Multivalent Antibodies

In another aspect, the isolated single domain antibody with an exposed C-terminal that has been modified to eliminate interaction with pre-existing antibodies can be a bispecific or multispecific antibody. The isolated single domain antibody with the modified C-terminus can be derivatized or linked to another functional molecule, e.g., another peptide or protein (e.g., another antibody or ligand for a receptor) to generate a bispecific molecule that binds to at least two different binding sites or target molecules.

Further clinical benefits may be provided by the binding of two or more antigens within one antibody (Morrison et al., (1997) Nature Biotech. 15:159-163; Alt et al. (1999) FEBS Letters 454:90-94; Zuo et al., (2000) Protein Engineering 13:361-367; Lu et al., (2004) JBC 279:2856-2865; Lu et al., (2005) JBC 280:19665-19672; Marvin et al., (2005) Acta Pharmacologica Sinica 26:649-658; Marvin et al., (2006) Curr Opin Drug Disc Develop 9:184-193; Shen et al., (2007) J Immun Methods 218:65-74; Wu et al., (2007) Nat Biotechnol. 11:1290-1297; Dimasi et al., (2009) J Mol Biol. 393:672-692; and Michaelson et al., (2009) mAbs 1:128-141.

A bispecific molecule can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

To create a bispecific molecule, the V_{H} or V_{L} with the modified C-terminus can be functionally linked (e.g., by chemical coupling, genetic fusion, non-covalent association or otherwise) to one or more other binding molecules, such as another single domain antibody, an antibody, antibody fragment, peptide or binding mimetic, such that a bispecific molecule results. The bispecific molecules can be prepared by conjugating the constituent binding specificities, using methods known in the art. For example, each binding specificity of the bispecific molecule can be generated separately and then conjugated to one another, for example, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., (1984) J. Exp. Med. 160:1686; Liu et al., (1985) Proc. Natl. Acad. Sci. USA 82:8648). Other methods include those described in Paulus (1985) Behring Ins. Mitt. No. 78:118-132; Brennan et al., (1985) Science 229:81-83), and Glennie et al., (1987) J. Immunol. 139: 2367-2375). Conjugating agents are SATA and sulfo-SMCC, both available from Pierce Chemical Co. (Rockford, Ill.).

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### Methods of Producing Single Domain Antibodies

In one embodiment, the isolated single domain antibody with an exposed C-terminal that has been modified to eliminate interaction with pre-existing antibodies can be obtained by providing a V_{HH} domain directed to a desired antigen, and (i) screening a library comprising heavy chain antibody sequences and/or V_{HH} sequences for sequences directed to the antigen; (ii) obtaining the heavy chain and/or V_{HH} sequences from the library; and (iii) modifying the V_{HH} sequences from the heavy chain and/or V_{HH} sequences by the addition or deletion of at least one amino acid reside at the exposed C-terminus.

In one embodiment, the single domain antibody with an exposed C-terminal that has been modified to eliminate interaction with pre-existing antibodies can further comprise the steps of (iv) subjecting the heavy chain antibody sequences and/or V_{HH} sequences to mutagenesis (e.g., random mutagenesis or site-directed mutagenesis), to increase the affinity and/or specificity of binding to the antigen; and (v) obtaining the obtaining the mutagenized single domain antibody from the heavy chain and/or V_{HH} sequences.

Generation of Single Domain Antibodies using Nucleotide and Amino Acid Substitutions

The isolated single domain antibody with an exposed C-terminal that has been modified to eliminate interaction with pre-existing antibodies can further comprise one or more amino acid or nucleotide modifications (e.g., alterations) that can be generated by a variety of known methods. Typically, isolated single domain antibodies with an exposed C-terminal are produced by recombinant methods. Moreover, because of the degeneracy of the genetic code, a variety of nucleic acid sequences can be used to encode each desired molecule.

Exemplary art recognized methods for making a nucleic acid molecule encoding an amino acid sequence variant of a starting molecule include, but are not limited to, preparation by site-directed (or oligonucleotide-mediated) mutagenesis, PCR mutagenesis, and cassette mutagenesis of an earlier prepared DNA encoding the molecule.

Site-directed mutagenesis is a preferred method for preparing substitution variants. This technique is well known in the art (see, e.g., Carter et al. Nucleic Acids Res. 13:4431-4443 (1985) and Kunkel et al., Proc. Natl. Acad. Sci. U.S.A 82:488 (1987)). Briefly, in carrying out site-directed mutagenesis of DNA, the parent DNA is altered by first hybridizing an oligonucleotide encoding the desired mutation to a single strand of such parent DNA. After hybridization, a DNA polymerase is used to synthesize an entire second strand, using the hybridized oligonucleotide as a primer, and using the single strand of the parent DNA as a template. Thus, the oligonucleotide encoding the desired mutation is incorporated in the resulting double-stranded DNA.

PCR mutagenesis is also suitable for making amino acid sequence variants of the starting molecule. See Higuchi, in PCR Protocols, pp. 177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the parent DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a polypeptide variant of the molecule can be determined, and a nucleic acid sequence encoding such amino acid sequence variant can be generated synthetically. In certain embodiments, the codon usage tables for various species are incorporated to modify the nucleotide sequence for optimization of protein expression. One skilled in the art would reference the various codon optimization charts depending the species of the cells in which the single domain antibody with an exposed C-terminal is to be expressed.

It will be understood by one of ordinary skill in the art that the isolated single domain antibody of the disclosure may further be modified such that they vary in amino acid sequence (e.g., from wild-type variants), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, i.e., a conservative substitutions, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Aside from amino acid substitutions, the present disclosure contemplates other modifications of the starting molecule amino acid sequence in order to generate molecules that reduce the interaction of pre-existing antibodies with the C-terminus of the isolated single domain antibody. In one embodiment, one may add one or more amino acid residues to the C-terminus of the isolated single domain antibody such that the modification to the C-terminus masks the interaction of pre-existing antibodies with the C-terminus of the isolated single domain antibody. In another embodiment, one may delete one or more amino acid residues from the C-terminus of the isolated single domain antibody such that the modification to the C-terminus eliminates the interaction of pre-existing antibodies with the C-terminus of the single domain antibody. The single domain antibodies with C-terminal modifications comprising one or more amino acid additions or deletions will preferably eliminate the pre-existing antibody response at least by about 5%, at least by about 10%, at least by about 20%, at least by about 30%, at least by about 40%, at least by about 50%, at least by about 60%, at least by about 70%, at least by about 80%, at least by about 90%, or at least by about 100%, compared with the a single domain antibody without the C-terminal modification.

In one embodiment, the disclosure relates to isolated single domain antibody with C-terminal modification where one or more natural amino acid residues are added to the C-terminus of the single domain antibody. In one embodiment, the disclosure relates to an isolated single domain antibody with C-terminal modification, where one or more non-natural amino acid residues are added to the C-terminus of the single domain antibody. In one embodiment, the disclosure relates to an isolated single domain antibody with C-terminal modification, one or more natural amino acid residues are deleted from the C-terminus of the single domain antibody.

### Library Construction and Screening

The methods disclosed in WO2006/09974 are incorporated herein by reference, in their entirety are used to generate libraries of the single domain antibodies. Oligonucleotides with randomized codons are created and incorporated into the V_{H} sequence. Each unique oligonucleotide is incorporated into a V_{H} gene, and the modified V_{H} genes constitute a library of sequences with slight variations. Typically, the oligonucleotides are designed such that the CDRs or loops of the V_{H} are randomized. For example, one, two or all three of V_{H} CDRs may be randomized. The V_{H} library is then cloned into an appropriate vector, depending on the type of library to be used, and the nucleic acid sequences are expressed as polypeptides. The library is screened for molecules that bind to the library polypeptides, typically by panning. The libraries may be phage display libraries, or other display libraries such as ribosome display and yeast display.

Once the V_{H}s or V_{L}s identified by the selection method have been isolated, they can be further manipulated to select for improved biophysical properties such as solubility, stability, monomericity, binding specificity, human origin or high expressability. This can be achieved by in vitro recombination techniques such as DNA shuffling or a staggered extension process. DNA shuffling involves cutting the nucleic acid sequence of first (donor) and second (acceptor) polypeptides, such as antibody fragments, into random fragments, then reassembling the random fragments by a PCR-like reaction. The reassembled fragments are then screened to select for the desired properties.

For example, one or more V_{H}s with high stability (donors) can be mixed with one or more V_{H}s lacking sufficient stability (acceptors) and subjected to DNA shuffling. This generates mutants of the acceptor V_{H}s which have incorporated stability residues from the donor V_{H}s. The newly stable mutants can be identified by the methods described herein, or through other evolutionary protein screening systems such as ribosome display, yeast display, bacterial cell display and phage display. Similarly, this technique can be used to transfer desirable traits such as solubility, monomericity, and high expression.

This technique may be used where both donor and acceptor V_{H}s have desirable properties, to produce a V_{H} with both properties. For example, an unstable donor V_{H} which binds to an important therapeutic or diagnostic ligand can be shuffled with a stable acceptor V_{H}. In order to ensure that new generated stable V_{H}s also have the ability to bind to the ligand, the screening system may involve a ligand binding step.

DNA shuffling may also be useful for humanizing non-human V_{H}s such as camelid heavy chain antibody variable domains and nurse shark and wobbegong shark variable domains, or non-human V_{L}s which bind to therapeutic targets. Human V_{H}s and V_{L}s with desirable properties such as solubility, stability, monomericity and high expressability may be used as donors. For example, one or more human V_{H}s with good stability (donors) can be mixed with one or more non-human therapeutic V_{H}s (acceptors) and subjected to DNA shuffling. This generates mutants of the acceptor V_{H}s which are both stable and humanized. The newly generated humanized and stable mutants can be identified by the methods described herein, or through other evolutionary protein screening systems such as ribosome display, yeast display, bacterial cell display and phage display. In a further example, the acceptor V_{H} could be a therapeutic V_{HH} (camelid heavy chain antibody variable domain).

Further, this technique is also useful for selecting desirable properties of polypeptides other than V_{H}s and V_{L}s. As discussed above, the donor polypeptide and the acceptor polypeptide may be both human, or the donor may be human and the acceptor non-human.

A possible approach for imparting solubility, monomericity, high expressability or stability to V_{H}s and V_{L}s may be through grafting CDRs onto acceptor V_{H}s and V_{L}s. Since CDRs are known to be involved in the solubility and stability of single-domain antibodies, and accordingly the grafting of these regions, such as the CDRs from V_{H}s and V_{L}s isolated by the methods described herein, may impart solubility and/or stability to acceptor V_{H}s and V_{L}s.

Monomeric human V_{H}s with different germline and overall sequences can be identified from a naïve human V_{H} phage display library using the selection method based on phage plaque size (See e.g., WO2006099747). The V_{H}s remain functional and monomeric following trypsin treatment at 37° C., weeks of incubations at 37° C. or months of storage at 4° C., have high thermal refolding efficiencies, are produced in good yields in E. coli and possess protein A binding activity. In addition, several monomeric human V_{L}s can be identified.

Such properties will also be manifested by V_{H}s from synthetic libraries that utilize the above V_{H}s as scaffolds. Similarly, libraries that utilize V_{L}s as scaffolds can be generated. Previously reported fully human V_{H}s with favorable biophysical properties were based on a single V germline sequence: DP-47 (Jespers et al. (2004), Nat. Biotechnol; 22, 1161-1165; and Jespers, et al. (2004) J. Mol. Biol. 337: 893-903). The observation that the monomeric human V_{H}s in this study stem from six different germline sequences including DP-47, demonstrates that stable V_{H}s are not restricted in terms of germline gene usage. In fact, it is very likely that we would have isolated monomeric V_{H}s of family and germline origins different from the ones we describe here had we not restricted our selection to a subset of V_{H}3 family V_{H}s with protein A binding activity.

Synthetic V_{H} libraries have been constructed on single scaffolds. Such an approach to repertoire generation is in sharp contrast to the natural, in vivo “approach” which utilizes a multiplicity of scaffolds. Based on the sequences reported here one can take advantage of the availability of the diverse set of V_{H}s and V_{L}s and create libraries which are based on multiple V_{H} and V_{L} scaffolds. Such libraries would be a better emulation of in vivo repertoires and therefore, would have a more optimal complexity. Such libraries would preferably consist of sub-libraries, where each sub-library is created by CDR3 randomization (and CDR1 and/or CDR2 randomization, if desired) on a single V_{H} or V_{L} scaffold without disrupting the parental CDR3 length.

The versatility of the present V_{H}s and V_{L}s is also beneficial in terms of choosing an optimal V_{H} or V_{L} framework for humanizing V_{HH}s, V_{H}s and V_{L}s which are specific to therapeutic targets. High affinity camelid V_{HH}s against therapeutic targets can be obtained from immune, non-immunized or synthetic V_{HH} libraries with relative ease and be subsequently subjected to humanization (CDR grafting, resurfacing, deimmunization) to remove possible V_{HH} immunogenicity, hence providing an alternative to human V_{H} library approach for production of therapeutic V_{H}s. Generating high affinity therapeutic V_{HS} by the latter approach may often require additional tedious and time consuming in vitro affinity maturation of the lead binder(s) selected from the primary synthetic human V_{H} libraries.

Nonhuman V_{H}S against therapeutic targets can be obtained from immune, non-immunized or synthetic V_{H} libraries with relative ease and be subsequently subjected to humanization (CDR grafting, resurfacing, deimmunization) to eliminate nonhuman V_{H}immunogenicity, hence providing an alternative to human V_{H} library approach for production of therapeutic V_{H}S.

Nonhuman V_{L}s against therapeutic targets can be obtained from immune, non-immunized or synthetic V_{HH} libraries with relative ease and be subsequently subjected to humanization (CDR grafting, resurfacing, deimmunization) to eliminate V_{HH} immunogenicity, hence providing an alternative to human V_{L} library approach for production of therapeutic V_{L}s.

Typically, stability pressure is required for selection of proteins with improved biophysical properties to ensure preferential selection of stable variants over unstable or less stable ones from a library population (Forrer et al. (1999) Curr. Opin. Struct. Biol. 9: 514-520; Waldo (2003) Curr. Opin. Chem. Biol. 7: 33-38; Jung et al. (1999). J. Mol. Biol. 294: 163-180; and Matsuura et al. (2003) FEBS Lett. 539: 24-28). For example, in a related work, heat treatment of V_{H} phage display libraries was required to select aggregation resistant V_{H}s. Examples of evolutionary selection approaches involving phage display include conventional phage display, selectively infective phage and the proteolysis approaches. In the first two approaches affinity selection is used to select stable species from a library, based on the assumption that stable proteins possess better binding properties for their ligand than the unstable ones. However, even with the additional inclusion of a stability selection step, these approaches may primarily enrich for higher affinity rather than for higher stability. A binding step requirement also limits the applicability of these approaches to proteins with known ligands. The third, proteolysis approach is based on the fact that stable proteins are generally compact and therefore are resistant to proteases whereas the unstable ones are not. The phage display format is engineered in such a way that the protease stability of the displayed protein translates to phage infectivity. Thus, when a variant phage display library is treated with a protease, only the phages displaying stable proteins retain their infectivity and can subsequently be selected by infecting an E. coli host. Since this approach is independent of ligand binding, it has general utility. However, even stable and well folded proteins have protease sensitive sites, e.g., loops and linkers, and this could sometimes hinder the selection of stable species in a proteolysis approach (Bai et at (2004). Eur. J. Biochem. 271: 1609-1614).

With the evolutionary approach disclosed in WO2006099747 and used herein, proteins with superior biophysical properties are simply identified by the naked eye. The approach does not require ligand binding, proteolysis or destabilization steps, and thus, avoids complications which may be encountered in the reported selection approaches. No requirement for a binding step also means that this approach has general utility. As an option, a binding step may be included to ensure that the selected proteins are functional.

Library construction and screening for standard antibodies and antibody fragments can also be used using techniques that are well known in the art, such as those described for example in WO2011/138391; WO2011/138392; and WO2012/022814. Also within the spirit of the disclosure are library construction and screening for antibody-like scaffolds, such as fibronections as described, for example, in WO2012/016245; WO2009/133208; WO2009/083804; US20110038866; and US20110275535.

### Methods of Manufacture

The isolated single domain antibody with an exposed C-terminal that has been modified to eliminate interaction with pre-existing antibodies are typically produced by recombinant expression. Nucleic acids encoding the molecules are inserted into expression vectors. The DNA segments encoding the molecules are operably linked to control sequences in the expression vector(s) that ensure their expression. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the single domain antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

E. coli is one prokaryotic host particularly useful for cloning the polynucleotides (e.g., DNA sequences) of the disclosure. Other microbial hosts suitable for use include bacilli, such as Bacillus subtilis, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture may also be used to express and produce the modified single domain antibodies of the present disclosure (e.g., polynucleotides encoding single domain antibodies or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., (1986) Immunol. Rev. 89:49), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. (See Co et al., (1992) J. Immunol. 148:1149).

Alternatively, coding sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the polynucleotide sequences of interest and expression control sequences can be transferred into the host cell by well-known methods, which vary depending on the type of cellular host. For example, chemically competent prokaryotic cells may be briefly heat-shocked, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, the modified single domain antibodies of the present disclosure can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure molecules of at least about 90 to 95% homogeneity are preferred, and 98 to 99% or more homogeneity most preferred, for pharmaceutical uses.

### Compositions

The single domain antibody with an exposed C-terminal that has been modified to eliminate interaction with pre-existing antibodies have in vivo therapeutic utilities. Accordingly, the present disclosure also provides compositions, e.g., a pharmaceutical composition, containing one or a combination of modified single domain antibodies (or variants, fusions, and conjugates thereof), formulated together with a pharmaceutically acceptable carrier. Pharmaceutical compositions of the disclosure also can be administered in combination therapy, i.e., combined with other agents. For example, the combination therapy can include a composition of the present disclosure with at least one or more additional therapeutic agents, such as anti-inflammatory agents, anti-cancer agents, and chemotherapeutic agents.

The pharmaceutical compositions of the disclosure can also be administered in conjunction with radiation therapy. Co-administration with other modified single domain antibodies are also encompassed by the disclosure.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

A modified single domain antibody can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer modified single domain antibody by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, a modified single domain antibody may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present disclosure include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient that can be combined with a carrier material to produce a single dosage form will vary depending upon the subject being treated, and the particular mode of administration. The amount of active ingredient that can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

Formulations of the present disclosure that are suitable for vaginal administration also include pessaries, tampons, creams, gels, pastes, foams or spray formulations containing such carriers as are known to be appropriate. Dosage forms for the topical or transdermal administration of modified single domain antibody compositions include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present disclosure are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the modified single domain antibody of the present disclosure, which may be used in a suitable hydrated form, and/or the pharmaceutical compositions of the present disclosure, are formulated into pharmaceutically acceptable dosage forms by conventional methods known to those of skill in the art.

Actual dosage levels of the active ingredients in the pharmaceutical compositions of the present disclosure may be varied so as to obtain an amount of the active ingredient that is effective to achieve the desired therapeutic response for a particular patient, mode of administration, and composition, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved. In general, a suitable daily dose of a compositions of the disclosure will be that amount of the compound that is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present disclosure to be administered alone, it is preferable to administer the compound as a pharmaceutical formulation (composition).

Therapeutic compositions can be administered with medical devices known in the art. For example, in a preferred embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the molecules of the disclosure can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the disclosures, as well as components of the invented molecules; p120 (Schreier et al. (1994) J. Biol. Chem. 269:9090); see also K. Keinanen (1994) FEBS Lett. 346:123; J. J. Killion (1994) Immunomethods 4:273. In one embodiment, the therapeutic compounds of the disclosure are formulated in liposomes; in a more preferred embodiment, the liposomes include a targeting moiety. In one embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

The composition must be sterile and fluid to the extent that the composition is deliverable by syringe. In addition to water, the carrier can be an isotonic buffered saline solution, ethanol, polyol (for example, glycerol, propylene glycol, and liquid polyethylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### Therapeutic and Diagnostic Applications

The single domain antibody with an exposed C-terminal that has been modified to eliminate interaction with pre-existing antibodies described herein may be constructed to bind any antigen or target of interest. Such targets include, but are not limited to, cluster domains, cell receptors, cell receptor ligands, growth factors, interleukins, protein allergens, bacteria, or viruses. The modified single domain antibody described herein may also be modified to have increased stability and half-life, as well as additional functional moieties. Accordingly, these molecules may be employed in place of antibodies in all areas in which antibodies are used, including in the research, therapeutic, and diagnostic fields. In addition, because these molecules possess solubility and stability properties superior to antibodies, the modified single domain antibody herein may be used under conditions that would destroy or inactivate antibody molecules.

For example, modified single domain antibody can be administered to cells in culture, e.g. in vitro or ex vivo, or in a subject, e.g., in vivo, to treat, prevent or diagnose a variety of disorders. The term “subject” as used herein includes human and non-human animals. Non-human animals includes all vertebrates, e.g., mammals and non-mammals, such as non-human primates, sheep, dogs, cats, cows, horses, chickens, amphibians, and reptiles. When the modified single domain antibody is administered together with another agent, the two can be administered in either order or simultaneously.

Also within the scope of the disclosure are kits comprising the compositions (e.g., the modified single domain antibody, variants, fusions, and conjugates thereof) of the disclosure and instructions for use. The kit can further contain a least one additional reagent, or one or more additional modified single domain antibodies of the disclosure. Kits typically include a label indicating the intended use of the contents of the kit. The term label includes any writing, or recorded material supplied on or with the kit, or which otherwise accompanies the kit.

As described above, the molecules of the present disclosure may be employed in all areas of the research, therapeutic, and diagnostic fields. Exemplary diseases/disorders which can be treated using the modified single domain antibody of the present disclosure (and variants, fusions, and conjugates thereof) include autoimmune disorders, cancers, infections, and other pathogenic indications.

Specific examples of autoimmune conditions in which the modified single domain antibody of the disclosure can be used include, but are not limited to, the following: multiple sclerosis and other demyelinating diseases; rheumatoid arthritis; inflammatory bowel disease; systemic lupus erythematosus; Type I diabetes; inflammatory skin disorders; Sjogren's Syndrome; and transplant rejection.

Specific examples of cancers in which the modified single domain antibody can be used include, but are not limited to, the following: lung; breast; prostate; bladder; melanoma; non-Hodgkin lymphoma; colon and rectal; pancreatic; endometrial; kidney; skin (non-melanoma); leukemia; and thyroid.

The modified single domain antibody can be used for the treatment of prevention of hyperproliferative diseases or cancer and the metastatic spread of cancers. Non-limiting examples of cancers include bladder, blood, bone, brain, breast, cartilage, colon kidney, liver, lung, lymph node, nervous tissue, ovary, pancreatic, prostate, skeletal muscle, skin, spinal cord, spleen, stomach, testes, thymus, thyroid, trachea, urogenital tract, ureter, urethra, uterus, or vaginal cancer. As described herein, angiogenesis-associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation and wound healing; telangiectasia psoriasis scleroderma, pyogenic granuloma, cororany collaterals, ischemic limb angiogenesis, corneal diseases, rubeosis, arthritis, diabetic neovascularization, fractures, vasculogenesis, hematopoiesis (see e.g., WO2005056764). Specific examples of infections in which the modified single domain antibody of the disclosure can be used include, but are not limited to, the following: cellular, fungal, bacterial, and viral.

Also included herein are methods for predicting whether pre-existing antibodies produce a pre-existing immune response with a single domain antibody comprising: contacting the single domain antibody with a human sample; determining whether a pre-existing antibody, if present in the human sample, binds to the single domain scaffold; and modifying the C-terminal region of single domain antibody by the deletion of at least one amino acid residue such that the C-terminal modification eliminates the interaction of the pre-existing antibody with the single domain antibody. The human sample is selected from the group consisting of blood and serum.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein.

### Examples

### Example 1

### Analysis for the Presence of Pre-Existing Antibodies in Human Serum Samples Directed Towards sdAb Scaffold

The aim of this assay is to detect response of pre-existing IgG in human blood samples from different donors to single domain antibodies (sdAb) and variation of this response by different C-terminal amino acid extensions. These extensions varied in amino acid composition and in length. Different scaffolds of human derived sdAb (heavy and light chain based) without any extension were tested for their immunogenic response with human sera. The overall goal was to find constructs without any or with low response to pre-existing antibodies.

### Samples:

Three human derived heavy chain sdAb scaffolds (HVHP, SEQ ID NOs: 8-13) and three human derived light chain sdAb scaffolds (LVHP, SEQ ID NOs: 2-7) without any extension were tested in the assay described below. These scaffolds were used to create a baseline to which the effect of extension and truncation at the C-terminus can be compared too. In a second set of experiments the C-terminal modifications and their effect on pre-existing antibody response were performed. The C-terminal modifications tested involved either the addition (extension) or deletion of amino acid residues from the C-terminus of either the V_{H} or V_{L} scaffold.

Scaffolds with C-terminal extensions were prepared which represent natural occurring amino acids in the linker region between the variable and the constant domain including but not limited to, Ala, Ala-Ala, Ala-Ser, Ala-Ser-Thr, Arg, Arg-Thr, Arg-Thr-Val, Gly-Gln, Gly-Gln-Pro, Ala-Ser-Thr-Lys-Pro (SEQ ID NO: 14). For the last variant listed, a non-naturally occurring Proline was added to prevent known clipping of C-terminal Lysine. In addition, an extension using an Ala-Ala and a Gly-Gly-Gly-Gly-Ser (SEQ ID NO: 15), stretch, which are normally used as a linker between single domains, was also tested. Additional extensions that were tested were Gly, Gly-Gly, and Gly-Ser.

| Workflow name | MW (Da) | Conc. (mg/ml) |
|---|---|---|
| HVHP430S in pTH296 | 13413 | 0.3 |
| HVHP426S in pTH296 | 12819 | 0.6 |
| HVHP421S in pTH296 | 13130 | 0.4 |
| HVLP325S in pTH296 | 11391 | 0.2 |
| HVLP351S in pTH296 | 11623 | 0.2 |
| HVLP3103S in pTH296 | 11620 | 0.05 |

### Serum Blood Samples:

20 serum samples from male donors and 20 samples from female donors were used. Hereby, known samples served as positive and negative control for each group with or without response.

| sample | |
|---|---|
| number | serum number |
| S01 | PC-male, IS4048 |
| S02 | NC-male, 10DS6446/HSer108 |
| S03 | male human serum 105 (10DS6443) |
| S04 | male human serum 106 (10DS6444) |
| S05 | male human serum 107 (10DS6445) |
| S06 | male human serum 109 (10DS6447) |
| S07 | male human serum 110 (10DS6448) |
| S08 | male human serum 111 (10DS6449) |
| S09 | male human serum 112 (10DS6450) |
| S10 | male human serum 113 (10DS6451) |
| S11 | male human serum 114 (10DS6452) |
| S12 | male human serum 115 (10DS6443) |
| S13 | male human serum 116 (10DS6454) |
| S14 | male human serum 117 (10DS6455) |
| S15 | male human serum 118 (10DS6456) |
| S16 | male human serum 119 (10DS6457) |
| S17 | male human serum 120 (IS4039) |
| S18 | male human serum 122 (10DS6460) |
| S19 | male human serum 124 (10DS6499) |
| S20 | male human serum 126 (10DS6501) |
| S21 | PC-female, 55019552 497 |
| S22 | NC-female, IS282 |
| S23 | female human serum 80 (10DS6462) |
| S24 | female human serum 81 (10DS6463) |
| S25 | female human serum 82 (10DS6464) |
| S26 | female human serum 83 (10DS6465) |
| S27 | female human serum 85 (10DS6467) |
| S28 | female human serum 86 (10DS6468) |
| S29 | female human serum 88 (10DS6470) |
| S30 | female human serum 89 (10DS6471) |
| S31 | female human serum 90 (10DS6472) |
| S32 | female human serum 91 (10DS6473) |
| S33 | female human serum 92 (10DS6474) |
| S34 | female human serum 93 (10DS6475) |
| S35 | female human serum 94 (10DS6476) |
| S36 | female human serum 95 (10DS6477) |
| S37 | female human serum 96 (10DS6478) |
| S38 | female human serum 97 (10DS6479) |
| S39 | female human serum 98 (10DS6480) |
| S40 | female human serum 99 (10DS6481) |

### Buffers:

| | |
|---|---|
| Coating buffer: | 1 × PBS, 5 mL 10 × PBS ad 50 mL H₂O |
| Washing buffer: | 1 × TBST, 2 bags of TBS-Tween |
| | 20 powder are dissolved in 2 L H₂O |
| Blocking buffer | 4 mL goat serum + 76 mL Superblock |
| Assay buffer: | 5.84 g NaCl ad 100 mL Lowcrossbuffer |

### Detection:

goat anti-hIgG-HRP (Fc) conjugated, 10.3 mg/mL (Sigma)

### Procedure:

Sandwich ELISA technique was used. SdAb constructs were directly immobilized on microtiter plate. Then human serum samples were added to the wells of the plate. If serum sample was captured by the sdAb construct it was detected by goat anti-human IgG coupled with horse radish peroxidase (HRP).

On the first day 100 μL coating solution (sdAbs) per well was added to the plate and incubated overnight at 4° C. The next day the plate was washed 3 times with 300 μL washing buffer per well. After blocking with 300 μL per well with blocking buffer for 2 h at room temperature the plate was washed 3 times with 300 μL washing buffer. Human serum samples were diluted 1:40 with assay buffer and then 100 μL per well of the diluted samples was added to the wells and incubated for 2 h at room temperature on a microtiter shaker with 300 rpm. Afterwards the plate was washed 3 times with 300 μL washing buffer. Then 100 μL per well detection antibody (1:100.000 dilution) was added to the wells and the plate was incubated 1 h at room temperature at 300 rpm. After washing plate 3 times with 300 μL per well washing buffer, 100 μL per well of TMB substrate solution was added to the plate and incubated for at least 10 min at room temperature at 300 rpm before adding 100 μL per well of TMB stop solution. The absorbance of individual wells was read at 450.

The values of the individual wells were normalized to the plate background (average of NC). Then the mean of normalized sample (duplicates) values were calculated. If the mean of normalized OD values was greater or equal to 2, it was set to positive immunogenic response (cut off point).

### Results:

Six single domain antibody scaffolds were tested for immunogenic response with human sera: −6 human sdAb scaffolds (3 VH and 3 VL).

TABLE 2
sdAb heavy and light chain scaffold response
to pre-existing antibodies

| sample_variant | # responder | % responder |
|---|---|---|
| HVHP430S | 25 | 62.5 |
| HVHP426S | 39 | 97.5 |
| HVHP421S | 20 | 50 |
| HVLP325S | 3 | 7.5 |
| HVLP351S | 9 | 22.5 |
| HVLP3103S | 3 | 7.5 |

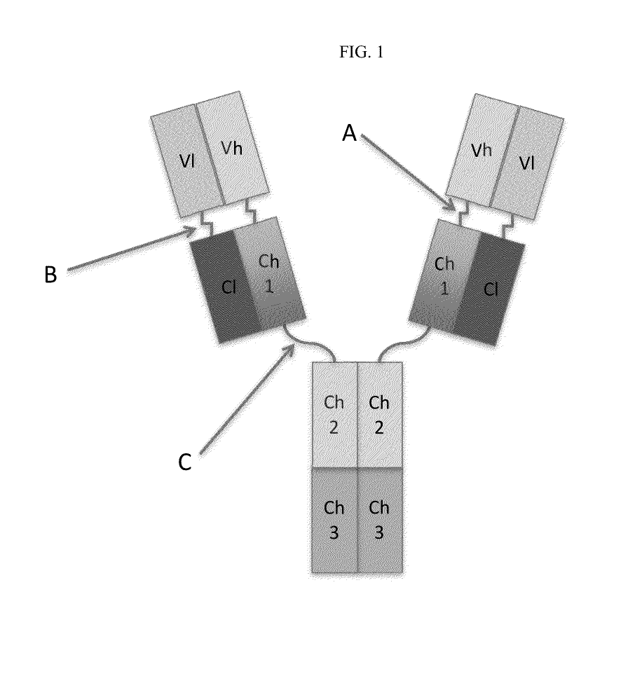

As can be seen in Table 2, the data for all three V_{H} scaffolds (designated HVHP430S, HVHP426S, HVHP421S), terminating at the natural occurring linker position −VSS, showed significant pre-existing immune response. In contrast, the V_{L} scaffolds (designated HVLP325S, HVLP351S and HVLP3103S), terminating at the natural occurring linker position −TKVEIK (for HVLP325S, HVLP351S) or −TKVTVL (HVLP3103S), respectively, did not show a pre-existing immune response. This led to the conclusion that the position of termination (cleavage) of the V_{H} or V_{L} scaffold is critical for the observed pre-existing immune response. It is Applicants position that this response can be altered by terminating (cleaving) at different amino acid positions in the linker, or by masking the exposed amino acids residue of the natural linker with additional amino acids not naturally part of the linker sequences between the variable and constant domains. To evaluate Applicants position, a second set of V_{H} and V_{L} scaffolds were prepared that only varies the C-terminus of the V_{H} or V_{L} scaffolds while the remaining V_{H} or V_{L} scaffold sequences were kept identical. Both truncations (deletions) of amino acids, as well as maintaining amino acids along the natural occurring linker sequences, were tested. In addition, masking the exposed linker with different amino acids that were not part of the natural linker sequence was also tested by adding amino acid residues to the C-terminus of the V_{H} or V_{L} scaffolds. The hypothesis being that the pre-existing immune response to isolated V_{H} or V_{L} domains in the serum of healthy donors is part of a natural clearing mechanism to remove degraded antibodies from circulation. The antibody epitope recognized by these pre-existing antibodies becomes exposed when the C-terminus of the normally hidden linker regions between the VH-CH1 (FIG. 1 cleavage position A) or VL-CL (FIG. 1 cleavage position B) is cleaved. This response is likely to be altered by adding or removing selective amino acids from the C-terminus of the VH or VL scaffolds, respectively. Furthermore, masking the C-terminus with additional amino acids not normally present the linker sequence will also reduce the response to pre-existing antibodies. The same mechanism is likely to give rise to pre-existing immune response directed against the exposed C-terminus in other antibody fragments such as Fab's or scFv's. Again, as in the case of isolated VH only or VL only antibody fragments, this response is likely to be masked by terminating (cleaving) at selective amino acids within the natural linker regions (e.g., between CH1-CH2 for a Fab fragment (FIG. 1 cleavage position C), or at the C-terminus of a scFv); or by masking with additional amino acids not normally present the natural linker sequence. Table 3 shows a number of V_{H} and V_{L} single domain antibody sequences that have been generated from Geneart to examine the effect of C-terminal modifications on pre-existing antibody response. The V_{H} and V_{L} single domain antibody sequences include the PhoA leader sequence (SEQ ID NO: 1), which is removed after expression and purification leaving the V_{H} and V_{L} single domain antibody sequences alone (SEQ ID NOs: 2-13), described above.

TABLE 3
V_{H} and V_{L} single domain antibody sequences including the
PhoA leader sequence (SEQ ID NO: 1) generated to examine
the effect of C-terminal modifications on pre-existing
immune response.

| Sequence | Modification |
|---|---|
| HVLP335 | |
| MKQSTIALALLPLLFTPVTKAEIVMTQSPATLSLSPGERATLSC | WT HVLP335 |
| RASQSVSSSSLAWYQQKPGQAPRLLIYGTSNRATGIPDRFSGS | |
| GSGTHFTLTINRLEPGDFAVYYCQQYGSSPRTFGQGTKVEIK | |
| (SEQ ID NO: 16) | |
| MKQSTIALALLPLLFTPVTKAEIVMTQSPATLSLSPGERATLSC | A |
| RASQSVSSSSLAWYQQKPGQAPRLLIYGTSNRATGIPDRFSGS | |
| GSGTHFTLTINRLEPGDFAVYYCQQYGSSPRTFGQGTKVEIKA | |
| (SEQ ID NO: 17) | |
| MKQSTIALALLPLLFTPVTKAEIVMTQSPATLSLSPGERATLSC | AA |
| RASQSVSSSSLAWYQQKPGQAPRLLIYGTSNRATGIPDRFSGS | |
| GSGTHFTLTINRLEPGDFAVYYCQQYGSSPRTFGQGTKVEIKA | |
| A (SEQ ID NO: 18) | |
| MKQSTIALALLPLLFTPVTKAEIVMTQSPATLSLSPGERATLSC | AS |
| RASQSVSSSSLAWYQQKPGQAPRLLIYGTSNRATGIPDRFSGS | |
| GSGTHFTLTINRLEPGDFAVYYCQQYGSSPRTFGQGTKVEIKA | |
| S (SEQ ID NO: 19) | |
| MKQSTIALALLPLLFTPVTKAEIVMTQSPATLSLSPGERATLSC | AST |
| RASQSVSSSSLAWYQQKPGQAPRLLIYGTSNRATGIPDRFSGS | |
| GSGTHFTLTINRLEPGDFAVYYCQQYGSSPRTFGQGTKVEIKA | |
| ST (SEQ ID NO: 20) | |
| MKQSTIALALLPLLFTPVTKAEIVMTQSPATLSLSPGERATLSC | ASTKP |
| RASQSVSSSSLAWYQQKPGQAPRLLIYGTSNRATGIPDRFSGS | |
| GSGTHFTLTINRLEPGDFAVYYCQQYGSSPRTFGQGTKVEIKA | |
| STKP (SEQ ID NO: 21) | |
| MKQSTIALALLPLLFTPVTKAEIVMTQSPATLSLSPGERATLSC | GGGGS |
| RASQSVSSSSLAWYQQKPGQAPRLLIYGTSNRATGIPDRFSGS | |
| GSGTHFTLTINRLEPGDFAVYYCQQYGSSPRTFGQGTKVEIKG | |
| GGGS (SEQ ID NO: 22) | |
| MKQSTIALALLPLLFTPVTKAEIVMTQSPATLSLSPGERATLSC | G |
| RASQSVSSSSLAWYQQKPGQAPRLLIYGTSNRATGIPDRFSGS | |
| GSGTHFTLTINRLEPGDFAVYYCQQYGSSPRTFGQGTKVEIKG | |
| (SEQ ID NO: 23) | |
| MKQSTIALALLPLLFTPVTKAEIVMTQSPATLSLSPGERATLSC | GG |
| RASQSVSSSSLAWYQQKPGQAPRLLIYGTSNRATGIPDRFSGS | |
| GSGTHFTLTINRLEPGDFAVYYCQQYGSSPRTFGQGTKVEIKG | |
| G (SEQ ID NO: 24) | |
| MKQSTIALALLPLLFTPVTKAEIVMTQSPATLSLSPGERATLSC | GS |
| RASQSVSSSSLAWYQQKPGQAPRLLIYGTSNRATGIPDRFSGS | |
| GSGTHFTLTINRLEPGDFAVYYCQQYGSSPRTFGQGTKVEIKG | |
| S ((SEQ ID NO: 25) | |
| HVLP3103S | |
| MKQSTIALALLPLLFTPVTKAETTLTQSPGTLSLSPGERATLSC | WT |
| RASQSVRNNLAWYQQRPGQAPRLLCYGASTRATGIPARFSCS | HVLP3103S |
| GSGTDFTLTISSLQVEDVAVYYCQQYYTTPKTFGQGTKVEIK | |
| (SEQ ID NO: 26) | |
| MKQSTIALALLPLLFTPVTKAETTLTQSPGTLSLSPGERATLSC | A |
| RASQSVRNNLAWYQQRPGQAPRLLCYGASTRATGIPARFSCS | |
| GSGTDFTLTISSLQVEDVAVYYCQQYYTTPKTFGQGTKVEIKA | |
| (SEQ ID NO: 27) | |
| MKQSTIALALLPLLFTPVTKAETTLTQSPGTLSLSPGERATLSC | AA |
| RASQSVRNNLAWYQQRPGQAPRLLCYGASTRATGIPARFSCS | |
| GSGTDFTLTISSLQVEDVAVYYCQQYYTTPKTFGQGTKVEIKA | |
| A (SEQ ID NO: 28) | |
| MKQSTIALALLPLLFTPVTKAETTLTQSPGTLSLSPGERATLSC | AS |
| RASQSVRNNLAWYQQRPGQAPRLLCYGASTRATGIPARFSCS | |
| GSGTDFTLTISSLQVEDVAVYYCQQYYTTPKTFGQGTKVEIKA | |
| S (SEQ ID NO: 29) | |
| MKQSTIALALLPLLFTPVTKAETTLTQSPGTLSLSPGERATLSC | AST |
| RASQSVRNNLAWYQQRPGQAPRLLCYGASTRATGIPARFSCS | |
| GSGTDFTLTISSLQVEDVAVYYCQQYYTTPKTFGQGTKVEIKA | |
| ST (SEQ ID NO: 30) | |
| MKQSTIALALLPLLFTPVTKAETTLTQSPGTLSLSPGERATLSC | ASTKP |
| RASQSVRNNLAWYQQRPGQAPRLLCYGASTRATGIPARFSCS | |
| GSGTDFTLTISSLQVEDVAVYYCQQYYTTPKTFGQGTKVEIKA | |
| STKP (SEQ ID NO: 31) | |
| MKQSTIALALLPLLFTPVTKAETTLTQSPGTLSLSPGERATLSC | GGGGS |
| RASQSVRNNLAWYQQRPGQAPRLLCYGASTRATGIPARFSCS | |
| GSGTDFTLTISSLQVEDVAVYYCQQYYTTPKTFGQGTKVEIKG | |
| GGGS (SEQ ID NO: 32) | |
| MKQSTIALALLPLLFTPVTKAETTLTQSPGTLSLSPGERATLSC | G |
| RASQSVRNNLAWYQQRPGQAPRLLCYGASTRATGIPARFSCS | |
| GSGTDFTLTISSLQVEDVAVYYCQQYYTTPKTFGQGTKVEIKG | |
| (SEQ ID NO: 33) | |
| MKQSTIALALLPLLFTPVTKAETTLTQSPGTLSLSPGERATLSC | GG |
| RASQSVRNNLAWYQQRPGQAPRLLCYGASTRATGIPARFSCS | |
| GSGTDFTLTISSLQVEDVAVYYCQQYYTTPKTFGQGTKVEIKG | |
| G (SEQ ID NO: 34) | |
| MKQSTIALALLPLLFTPVTKAETTLTQSPGTLSLSPGERATLSC | GS |
| RASQSVRNNLAWYQQRPGQAPRLLCYGASTRATGIPARFSCS | |
| GSGTDFTLTISSLQVEDVAVYYCQQYYTTPKTFGQGTKVEIKG | |
| S (SEQ ID NO: 35) | |
| HVLP325 | |
| MKQSTIALALLPLLFTPVTKAEIVLTQSPTTLSLSPGERATLSCR | WT HVLP325 |
| ASQSVGRYLAWYQQRPGQAPRLLVFDTSNRAPGVPARFSGRG | |
| SGTLFTLTISSLEPEDSAVYFCQQRSSGLTFGGGTKVTVL | |
| (SEQ ID NO: 36) | |
| MKQSTIALALLPLLFTPVTKAEIVLTQSPTTLSLSPGERATLSCR | A |
| ASQSVGRYLAWYQQRPGQAPRLLVFDTSNRAPGVPARFSGRG | |
| SGTLFTLTISSLEPEDSAVYFCQQRSSGLTFGGGTKVTVLA | |
| (SEQ ID NO: 37) | |
| MKQSTIALALLPLLFTPVTKAEIVLTQSPTTLSLSPGERATLSCR | AA |
| ASQSVGRYLAWYQQRPGQAPRLLVFDTSNRAPGVPARFSGRG | |
| SGTLFTLTISSLEPEDSAVYFCQQRSSGLTFGGGTKVTVLAA | |
| (SEQ ID NO: 38) | |
| MKQSTIALALLPLLFTPVTKAEIVLTQSPTTLSLSPGERATLSCR | AS |
| ASQSVGRYLAWYQQRPGQAPRLLVFDTSNRAPGVPARFSGRG | |
| SGTLFTLTISSLEPEDSAVYFCQQRSSGLTFGGGTKVTVLAS | |
| (SEQ ID NO: 39) | |
| MKQSTIALALLPLLFTPVTKAEIVLTQSPTTLSLSPGERATLSCR | AST |
| ASQSVGRYLAWYQQRPGQAPRLLVFDTSNRAPGVPARFSGRG | |
| SGTLFTLTISSLEPEDSAVYFCQQRSSGLTFGGGTKVTVLAST | |
| (SEQ ID NO: 40) | |
| MKQSTIALALLPLLFTPVTKAEIVLTQSPTTLSLSPGERATLSCR | ASTKP |
| ASQSVGRYLAWYQQRPGQAPRLLVFDTSNRAPGVPARFSGRG | |
| SGTLFTLTISSLEPEDSAVYFCQQRSSGLTFGGGTKVTVLASTK | |
| P (SEQ ID NO: 41) | |
| MKQSTIALALLPLLFTPVTKAEIVLTQSPTTLSLSPGERATLSCR | GGGGS |
| ASQSVGRYLAWYQQRPGQAPRLLVFDTSNRAPGVPARFSGRG | |
| SGTLFTLTISSLEPEDSAVYFCQQRSSGLTFGGGTKVTVLGGGG | |
| S (SEQ ID NO: 42) | |
| MKQSTIALALLPLLFTPVTKAEIVLTQSPTTLSLSPGERATLSCR | G |
| ASQSVGRYLAWYQQRPGQAPRLLVFDTSNRAPGVPARFSGRG | |
| SGTLFTLTISSLEPEDSAVYFCQQRSSGLTFGGGTKVTVLG | |
| (SEQ ID NO: 43) | |
| MKQSTIALALLPLLFTPVTKAEIVLTQSPTTLSLSPGERATLSCR | GG |
| ASQSVGRYLAWYQQRPGQAPRLLVFDTSNRAPGVPARFSGRG | |
| SGTLFTLTISSLEPEDSAVYFCQQRSSGLTFGGGTKVTVLGG | |
| (SEQ ID NO: 44) | |
| MKQSTIALALLPLLFTPVTKAEIVLTQSPTTLSLSPGERATLSCR | GS |
| ASQSVGRYLAWYQQRPGQAPRLLVFDTSNRAPGVPARFSGRG | |
| SGTLFTLTISSLEPEDSAVYFCQQRSSGLTFGGGTKVTVLGS | |
| (SEQ ID NO: 45) | |
| HVLP325S | |
| MKQSTIALALLPLLFTPVTKAEIVLTQSPTTLSLSPGERATLSCR | WT HVLP325S |
| ASQSVGRYLAWYQQRPGQAPRLLCFDTSNRAPGVPARFSCRG | |
| SGTLFTLTISSLEPEDSAVYFCQQRSSGLTFGGGTKVTVL | |
| (SEQ ID NO: 46) | |
| MKQSTIALALLPLLFTPVTKAEIVLTQSPTTLSLSPGERATLSCR | A |
| ASQSVGRYLAWYQQRPGQAPRLLCFDTSNRAPGVPARFSCRG | |
| SGTLFTLTISSLEPEDSAVYFCQQRSSGLTFGGGTKVTVLA | |
| (SEQ ID NO: 47) | |
| MKQSTIALALLPLLFTPVTKAEIVLTQSPTTLSLSPGERATLSCR | AA |
| ASQSVGRYLAWYQQRPGQAPRLLCFDTSNRAPGVPARFSCRG | |
| SGTLFTLTISSLEPEDSAVYFCQQRSSGLTFGGGTKVTVLAA | |
| (SEQ ID NO: 48) | |
| MKQSTIALALLPLLFTPVTKAEIVLTQSPTTLSLSPGERATLSCR | AS |
| ASQSVGRYLAWYQQRPGQAPRLLCFDTSNRAPGVPARFSCRG | |
| SGTLFTLTISSLEPEDSAVYFCQQRSSGLTFGGGTKVTVLAS | |
| (SEQ ID NO: 49) | |
| MKQSTIALALLPLLFTPVTKAEIVLTQSPTTLSLSPGERATLSCR | AST |
| ASQSVGRYLAWYQQRPGQAPRLLCFDTSNRAPGVPARFSCRG | |
| SGTLFTLTISSLEPEDSAVYFCQQRSSGLTFGGGTKVTVLAST | |
| (SEQ ID NO: 50) | |
| MKQSTIALALLPLLFTPVTKAEIVLTQSPTTLSLSPGERATLSCR | ASTKP |
| ASQSVGRYLAWYQQRPGQAPRLLCFDTSNRAPGVPARFSCRG | |
| SGTLFTLTISSLEPEDSAVYFCQQRSSGLTFGGGTKVTVLASTK | |
| P (SEQ ID NO: 51) | |
| MKQSTIALALLPLLFTPVTKAEIVLTQSPTTLSLSPGERATLSCR | GGGGS |
| ASQSVGRYLAWYQQRPGQAPRLLCFDTSNRAPGVPARFSCRG | |
| SGTLFTLTISSLEPEDSAVYFCQQRSSGLTFGGGTKVTVLGGGG | |
| S (SEQ ID NO: 52) | |
| MKQSTIALALLPLLFTPVTKAEIVLTQSPTTLSLSPGERATLSCR | G |
| ASQSVGRYLAWYQQRPGQAPRLLCFDTSNRAPGVPARFSCRG | |
| SGTLFTLTISSLEPEDSAVYFCQQRSSGLTFGGGTKVTVLG | |
| (SEQ ID NO: 53) | |
| MKQSTIALALLPLLFTPVTKAEIVLTQSPTTLSLSPGERATLSCR | GG |
| ASQSVGRYLAWYQQRPGQAPRLLCFDTSNRAPGVPARFSCRG | |
| SGTLFTLTISSLEPEDSAVYFCQQRSSGLTFGGGTKVTVLGG | |
| (SEQ ID NO: 54) | |
| MKQSTIALALLPLLFTPVTKAEIVLTQSPTTLSLSPGERATLSCR | GS |
| ASQSVGRYLAWYQQRPGQAPRLLCFDTSNRAPGVPARFSCRG | |
| SGTLFTLTISSLEPEDSAVYFCQQRSSGLTFGGGTKVTVLGS | |
| (SEQ ID NO: 55) | |
| HVLP351 | |
| MKQSTIALALLPLLFTPVTKAEIVMTQSPVTLSLSPGERATLSC | WT HVLP351 |
| RASQSVGTSLAWYQQKPGQAPRLLIYDASNRATGISARFSGSG | |
| SGTDFTLTISSLEPEDFAVYYCQQRYNWPRTFGGGTKVTVL | |
| (SEQ ID NO: 56) | |
| MKQSTIALALLPLLFTPVTKAEIVMTQSPVTLSLSPGERATLSC | A |
| RASQSVGTSLAWYQQKPGQAPRLLIYDASNRATGISARFSGSG | |
| SGTDFTLTISSLEPEDFAVYYCQQRYNWPRTFGGGTKVTVLA | |
| (SEQ ID NO: 57) | |
| MKQSTIALALLPLLFTPVTKAEIVMTQSPVTLSLSPGERATLSC | AA |
| RASQSVGTSLAWYQQKPGQAPRLLIYDASNRATGISARFSGSG | |
| SGTDFTLTISSLEPEDFAVYYCQQRYNWPRTFGGGTKVTVLAA | |
| (SEQ ID NO: 58) | |
| MKQSTIALALLPLLFTPVTKAEIVMTQSPVTLSLSPGERATLSC | AS |
| RASQSVGTSLAWYQQKPGQAPRLLIYDASNRATGISARFSGSG | |
| SGTDFTLTISSLEPEDFAVYYCQQRYNWPRTFGGGTKVTVLAS | |
| (SEQ ID NO: 59) | |
| MKQSTIALALLPLLFTPVTKAEIVMTQSPVTLSLSPGERATLSC | AST |
| RASQSVGTSLAWYQQKPGQAPRLLIYDASNRATGISARFSGSG | |
| SGTDFTLTISSLEPEDFAVYYCQQRYNWPRTFGGGTKVTVLAS | |
| T (SEQ ID NO: 60) | |
| MKQSTIALALLPLLFTPVTKAEIVMTQSPVTLSLSPGERATLSC | ASTKP |
| RASQSVGTSLAWYQQKPGQAPRLLIYDASNRATGISARFSGSG | |
| SGTDFTLTISSLEPEDFAVYYCQQRYNWPRTFGGGTKVTVLAS | |
| TKP (SEQ ID NO: 61) | |
| MKQSTIALALLPLLFTPVTKAEIVMTQSPVTLSLSPGERATLSC | GGGGS |
| RASQSVGTSLAWYQQKPGQAPRLLIYDASNRATGISARFSGSG | |
| SGTDFTLTISSLEPEDFAVYYCQQRYNWPRTFGGGTKVTVLGG | |
| GGS (SEQ ID NO: 62) | |
| MKQSTIALALLPLLFTPVTKAEIVMTQSPVTLSLSPGERATLSC | G |
| RASQSVGTSLAWYQQKPGQAPRLLIYDASNRATGISARFSGSG | |
| SGTDFTLTISSLEPEDFAVYYCQQRYNWPRTFGGGTKVTVLG | |
| (SEQ ID NO: 63) | |
| MKQSTIALALLPLLFTPVTKAEIVMTQSPVTLSLSPGERATLSC | GG |
| RASQSVGTSLAWYQQKPGQAPRLLIYDASNRATGISARFSGSG | |
| SGTDFTLTISSLEPEDFAVYYCQQRYNWPRTFGGGTKVTVLGG | |
| (SEQ ID NO: 64) | |
| MKQSTIALALLPLLFTPVTKAEIVMTQSPVTLSLSPGERATLSC | GS |
| RASQSVGTSLAWYQQKPGQAPRLLIYDASNRATGISARFSGSG | |
| SGTDFTLTISSLEPEDFAVYYCQQRYNWPRTFGGGTKVTVLGS | |
| (SEQ ID NO: 65) | |
| HVLP351S | |
| MKQSTIALALLPLLFTPVTKAEIVMTQSPVTLSLSPGERATLSC | WT HVLP351S |
| RASQSVGTSLAWYQQKPGQAPRLLCYDASNRATGISARFSCS | |
| GSGTDFTLTISSLEPEDFAVYYCQQRYNWPRTFGGGTKVTVL | |
| (SEQ ID NO: 66) | |
| MKQSTIALALLPLLFTPVTKAEIVMTQSPVTLSLSPGERATLSC | A |
| RASQSVGTSLAWYQQKPGQAPRLLCYDASNRATGISARFSCS | |
| GSGTDFTLTISSLEPEDFAVYYCQQRYNWPRTFGGGTKVTVLA | |
| (SEQ ID NO: 67) | |
| MKQSTIALALLPLLFTPVTKAEIVMTQSPVTLSLSPGERATLSC | AA |
| RASQSVGTSLAWYQQKPGQAPRLLCYDASNRATGISARFSCS | |
| GSGTDFTLTISSLEPEDFAVYYCQQRYNWPRTFGGGTKVTVLA | |
| A (SEQ ID NO: 68) | |
| MKQSTIALALLPLLFTPVTKAEIVMTQSPVTLSLSPGERATLSC | AS |
| RASQSVGTSLAWYQQKPGQAPRLLCYDASNRATGISARFSCS | |
| GSGTDFTLTISSLEPEDFAVYYCQQRYNWPRTFGGGTKVTVLA | |
| S (SEQ ID NO: 69) | |
| MKQSTIALALLPLLFTPVTKAEIVMTQSPVTLSLSPGERATLSC | AST |
| RASQSVGTSLAWYQQKPGQAPRLLCYDASNRATGISARFSCS | |
| GSGTDFTLTISSLEPEDFAVYYCQQRYNWPRTFGGGTKVTVLA | |
| ST (SEQ ID NO: 70) | |
| MKQSTIALALLPLLFTPVTKAEIVMTQSPVTLSLSPGERATLSC | ASTKP |
| RASQSVGTSLAWYQQKPGQAPRLLCYDASNRATGISARFSCS | |
| GSGTDFTLTISSLEPEDFAVYYCQQRYNWPRTFGGGTKVTVLA | |
| STKP (SEQ ID NO: 71) | |
| MKQSTIALALLPLLFTPVTKAEIVMTQSPVTLSLSPGERATLSC | GGGGS |
| RASQSVGTSLAWYQQKPGQAPRLLCYDASNRATGISARFSCS | |
| GSGTDFTLTISSLEPEDFAVYYCQQRYNWPRTFGGGTKVTVLG | |
| GGGS (SEQ ID NO: 72) | |
| MKQSTIALALLPLLFTPVTKAEIVMTQSPVTLSLSPGERATLSC | G |
| RASQSVGTSLAWYQQKPGQAPRLLCYDASNRATGISARFSCS | |
| GSGTDFTLTISSLEPEDFAVYYCQQRYNWPRTFGGGTKVTVLG | |
| (SEQ ID NO: 73) | |
| MKQSTIALALLPLLFTPVTKAEIVMTQSPVTLSLSPGERATLSC | GG |
| RASQSVGTSLAWYQQKPGQAPRLLCYDASNRATGISARFSCS | |
| GSGTDFTLTISSLEPEDFAVYYCQQRYNWPRTFGGGTKVTVLG | |
| G (SEQ ID NO: 74) | |
| MKQSTIALALLPLLFTPVTKAEIVMTQSPVTLSLSPGERATLSC | GS |
| RASQSVGTSLAWYQQKPGQAPRLLCYDASNRATGISARFSCS | |
| GSGTDFTLTISSLEPEDFAVYYCQQRYNWPRTFGGGTKVTVLG | |
| S (SEQ ID NO: 75) | |
| HVHP426 | |
| MKQSTIALALLPLLFTPVTKAQVQLVQSGGGVVQPGRSLRLSC | WT HVHP426 |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSS (SEQ ID NO: 76) | |
| MKQSTIALALLPLLFTPVTKAQVQLVQSGGGVVQPGRSLRLSC | A |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSA (SEQ ID NO: 77) | |
| MKQSTIALALLPLLFTPVTKAQVQLVQSGGGVVQPGRSLRLSC | AA |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSAA (SEQ ID NO: 78) | |
| MKQSTIALALLPLLFTPVTKAQVQLVQSGGGVVQPGRSLRLSC | AS |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSAS (SEQ ID NO: 79) | |
| MKQSTIALALLPLLFTPVTKAQVQLVQSGGGVVQPGRSLRLSC | AST |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSAST (SEQ ID NO: 80) | |
| MKQSTIALALLPLLFTPVTKAQVQLVQSGGGVVQPGRSLRLSC | ASTKP |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSASTKP (SEQ ID NO: 81) | |
| MKQSTIALALLPLLFTPVTKAQVQLVQSGGGVVQPGRSLRLSC | GGGGS |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSGGGGS (SEQ ID NO: 82) | |
| MKQSTIALALLPLLFTPVTKAQVQLVQSGGGVVQPGRSLRLSC | G |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSG (SEQ ID NO: 83) | |
| MKQSTIALALLPLLFTPVTKAQVQLVQSGGGVVQPGRSLRLSC | GG |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSGG (SEQ ID NO: 84) | |
| MKQSTIALALLPLLFTPVTKAQVQLVQSGGGVVQPGRSLRLSC | GS |
| AASGFIVDGYAMHWVRQAPGQGLEWVSVTNNGGSTSYADSV | |
| KGRFTISRDNSKNTVYLQMNSLRAEDTAVYYCARQSITGPTG | |
| AFDIWGQGTMVTVSSGS (SEQ ID NO: 85) | |
| HVHP426S | |
| MKQSTIALALLPLLFTPVTKAQVQLVQSGGGVVQPGRSLRLSC | WT HVHP426S |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSS (SEQ ID NO: 86) | |
| MKQSTIALALLPLLFTPVTKAQVQLVQSGGGVVQPGRSLRLSC | A |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSA (SEQ ID NO: 87) | |
| MKQSTIALALLPLLFTPVTKAQVQLVQSGGGVVQPGRSLRLSC | AA |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSAA (SEQ ID NO: 88) | |
| MKQSTIALALLPLLFTPVTKAQVQLVQSGGGVVQPGRSLRLSC | AS |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSAS (SEQ ID NO: 89) | |
| MKQSTIALALLPLLFTPVTKAQVQLVQSGGGVVQPGRSLRLSC | AST |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSAST (SEQ ID NO: 90) | |
| MKQSTIALALLPLLFTPVTKAQVQLVQSGGGVVQPGRSLRLSC | ASTKP |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSASTKP (SEQ ID NO: 91) | |
| MKQSTIALALLPLLFTPVTKAQVQLVQSGGGVVQPGRSLRLSC | GGGGS |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSGGGGS (SEQ ID NO: 92) | |
| MKQSTIALALLPLLFTPVTKAQVQLVQSGGGVVQPGRSLRLSC | G |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSG (SEQ ID NO: 93) | |
| MKQSTIALALLPLLFTPVTKAQVQLVQSGGGVVQPGRSLRLSC | GS |
| AASGFIVDGYAMHWVRQAPGQGLEWVCVTNNGGSTSYADS | |
| VKGRFTCSRDNSKNTVYLQMNSLRAEDTAVYYCARQSITGPT | |
| GAFDIWGQGTMVTVSSGS (SEQ ID NO: 95) | |
| HVHP420 | |
| MKQSTIALALLPLLFTPVTKAQVQLVESGGGLVKPGGSLRLSC | WT HVHP420 |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSS (SEQ ID NO: 96) | |
| MKQSTIALALLPLLFTPVTKAQVQLVESGGGLVKPGGSLRLSC | A |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSA (SEQ ID NO: 97) | |
| MKQSTIALALLPLLFTPVTKAQVQLVESGGGLVKPGGSLRLSC | AA |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSAA (SEQ ID NO: 98) | |
| MKQSTIALALLPLLFTPVTKAQVQLVESGGGLVKPGGSLRLSC | AS |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSAS (SEQ ID NO: 99) | |
| MKQSTIALALLPLLFTPVTKAQVQLVESGGGLVKPGGSLRLSC | AST |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSAST (SEQ ID NO: 100) | |
| MKQSTIALALLPLLFTPVTKAQVQLVESGGGLVKPGGSLRLSC | ASTKP |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSASTKP (SEQ ID NO: 101) | |
| MKQSTIALALLPLLFTPVTKAQVQLVESGGGLVKPGGSLRLSC | GGGGS |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSGGGGS (SEQ ID NO: 102) | |
| MKQSTIALALLPLLFTPVTKAQVQLVESGGGLVKPGGSLRLSC | G |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSG (SEQ ID NO: 103) | |
| MKQSTIALALLPLLFTPVTKAQVQLVESGGGLVKPGGSLRLSC | GG |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSGG (SEQ ID NO: 104) | |
| MKQSTIALALLPLLFTPVTKAQVQLVESGGGLVKPGGSLRLSC | GS |
| AASGFTFSNAWMTWVRQAPGKGLEWVGRIKTKTDGGTTDYA | |
| APVKGRFTISRDDSKNTLYLQMNSLKTEDTAVYYCTTDRDHS | |
| SGSWGQGTLVTVSSGS (SEQ ID NO: 105) | |
| HVHM81 | |
| MKQSTIALALLPLLFTPVTKAEVQLVQSGGGLVQPGRSLRLSC | WT HVHM81 |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSS (SEQ ID NO: 106) | |
| MKQSTIALALLPLLFTPVTKAEVQLVQSGGGLVQPGRSLRLSC | A |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSA (SEQ ID NO: 107) | |
| MKQSTIALALLPLLFTPVTKAEVQLVQSGGGLVQPGRSLRLSC | AA |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSAA (SEQ ID NO: 108) | |
| MKQSTIALALLPLLFTPVTKAEVQLVQSGGGLVQPGRSLRLSC | AS |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSAS (SEQ ID NO: 109) | |
| MKQSTIALALLPLLFTPVTKAEVQLVQSGGGLVQPGRSLRLSC | AST |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSAST (SEQ ID NO: 110) | |
| MKQSTIALALLPLLFTPVTKAEVQLVQSGGGLVQPGRSLRLSC | ASTKP |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSASTKP (SEQ ID NO: 111) | |
| MKQSTIALALLPLLFTPVTKAEVQLVQSGGGLVQPGRSLRLSC | GGGGS |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSGGGGS (SEQ ID NO: 112) | |
| MKQSTIALALLPLLFTPVTKAEVQLVQSGGGLVQPGRSLRLSC | G |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSG (SEQ ID NO: 113) | |
| MKQSTIALALLPLLFTPVTKAEVQLVQSGGGLVQPGRSLRLSC | GG |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSGG (SEQ ID NO: 114) | |
| MKQSTIALALLPLLFTPVTKAEVQLVQSGGGLVQPGRSLRLSC | GS |
| AASGFTFDDYAMHWVRQAPGKGLEWVSGISGSGASTYYADS | |
| VKGRFTISRDNSKNTLYLQMNSLRAGDTALYYCARQSITGPTG | |
| AFDVWGQGTMVTVSSGS (SEQ ID NO: 115) | |
| HVHP421S | |
| MKQSTIALALLPLLFTPVTKAQLQLQESGGGVVQPGRSLRLSC | WT HVHP421S |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSS (SEQ ID NO: 116) | |
| MKQSTIALALLPLLFTPVTKAQLQLQESGGGVVQPGRSLRLSC | A |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSA (SEQ ID NO: 117) | |
| MKQSTIALALLPLLFTPVTKAQLQLQESGGGVVQPGRSLRLSC | AA |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSAA (SEQ ID NO: 118) | |
| MKQSTIALALLPLLFTPVTKAQLQLQESGGGVVQPGRSLRLSC | AS |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSAS (SEQ ID NO: 119) | |
| MKQSTIALALLPLLFTPVTKAQLQLQESGGGVVQPGRSLRLSC | AST |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSAST (SEQ ID NO: 120) | |
| MKQSTIALALLPLLFTPVTKAQLQLQESGGGVVQPGRSLRLSC | ASTKP |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSASTKP (SEQ ID NO: 121) | |
| MKQSTIALALLPLLFTPVTKAQLQLQESGGGVVQPGRSLRLSC | GGGGS |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSGGGGS (SEQ ID NO: 122) | |
| MKQSTIALALLPLLFTPVTKAQLQLQESGGGVVQPGRSLRLSC | G |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSG (SEQ ID NO: 123) | |
| MKQSTIALALLPLLFTPVTKAQLQLQESGGGVVQPGRSLRLSC | GG |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSGG (SEQ ID NO: 124) | |
| MKQSTIALALLPLLFTPVTKAQLQLQESGGGVVQPGRSLRLSC | GS |
| AASGFTFSSYAMSWVRQAPGKGLEWVCAISGSGGSTYYADSV | |
| KGRFTCSRDNSKNTLYLQMNSLRAEDTAVYYCAKDGKGGSS | |
| GYDHPDYWGQGTLVTVSSGS (SEQ ID NO: 125) | |
| HVHP430S | |
| MKQSTIALALLPLLFTPVTKAQVQLVESGGGLIKPGGSLRLSC | WT HVHP430S |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSS (SEQ ID NO: 126) | |
| MKQSTIALALLPLLFTPVTKAQVQLVESGGGLIKPGGSLRLSC | A |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSA (SEQ ID NO: 127) | |
| MKQSTIALALLPLLFTPVTKAQVQLVESGGGLIKPGGSLRLSC | AA |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSAA (SEQ ID NO: 128) | |
| MKQSTIALALLPLLFTPVTKAQVQLVESGGGLIKPGGSLRLSC | AS |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSAS (SEQ ID NO: 129) | |
| MKQSTIALALLPLLFTPVTKAQVQLVESGGGLIKPGGSLRLSC | AST |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSAST (SEQ ID NO: 130) | |
| MKQSTIALALLPLLFTPVTKAQVQLVESGGGLIKPGGSLRLSC | ASTKP |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSASTKP (SEQ ID NO: 131) | |
| MKQSTIALALLPLLFTPVTKAQVQLVESGGGLIKPGGSLRLSC | GGGGS |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSGGGGS (SEQ ID NO: 132) | |
| MKQSTIALALLPLLFTPVTKAQVQLVESGGGLIKPGGSLRLSC | G |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSG (SEQ ID NO: 133) | |
| MKQSTIALALLPLLFTPVTKAQVQLVESGGGLIKPGGSLRLSC | GG |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSGG (SEQ ID NO: 134) | |
| MKQSTIALALLPLLFTPVTKAQVQLVESGGGLIKPGGSLRLSC | GS |
| AASGFTFSNYAMSWVRQAPGKGLEWVCAISSSGGSTYYADSV | |
| KGRFTCSRDNSKNTVYLQMNSLRAEDTAVYYCVREEYRCSGT | |
| SCPGAFDIWGQGTMVTVSSGS (SEQ ID NO: 135) | |
| MKQSTIALALLPLLFTPVTKAEIVMTQSPATLSLSPGERATLSC | SEQ ID NO: 16 |
| RASQSVSSSSLAWYQQKPGQAPRLLIYGTSNRATGIPDRFSGS | −1 C-term aa |
| GSGTHFTLTINRLEPGDFAVYYCQQYGSSPRTFGQGTKVEI | |
| (SEQ ID NO: 136) | |
| MKQSTIALALLPLLFTPVTKAEIVMTQSPATLSLSPGERATLSC | SEQ ID NO: 16 |
| RASQSVSSSSLAWYQQKPGQAPRLLIYGTSNRATGIPDRFSGS | −2 C-term aa |
| GSGTHFTLTINRLEPGDFAVYYCQQYGSSPRTFGQGTKVE | |
| (SEQ ID NO: 137) | |
| MKQSTIALALLPLLFTPVTKAEIVMTQSPATLSLSPGERATLSC | SEQ ID NO: 16 |
| RASQSVSSSSLAWYQQKPGQAPRLLIYGTSNRATGIPDRFSGS | −3 C-term aa |
| GSGTHFTLTINRLEPGDFAVYYCQQYGSSPRTFGQGTKV | |
| (SEQ ID NO: 138) | |
| MKQSTIALALLPLLFTPVTKAETTLTQSPGTLSLSPGERATLSC | SEQ ID NO: 26 |
| RASQSVRNNLAWYQQRPGQAPRLLCYGASTRATGIPARFSCS | −1 C-term aa |
| GSGTDFTLTISSLQVEDVAVYYCQQYYTTPKTFGQGTKVEI | |
| (SEQ ID NO: 139) | |
| MKQSTIALALLPLLFTPVTKAETTLTQSPGTLSLSPGERATLSC | SEQ ID NO: 26 |
| RASQSVRNNLAWYQQRPGQAPRLLCYGASTRATGIPARFSCS | −2 C-term aa |
| GSGTDFTLTISSLQVEDVAVYYCQQYYTTPKTFGQGTKVE | |
| (SEQ ID NO: 140) | |
| MKQSTIALALLPLLFTPVTKAETTLTQSPGTLSLSPGERATLSC | SEQ ID NO: 26 |
| RASQSVRNNLAWYQQRPGQAPRLLCYGASTRATGIPARFSCS | −3 C-term aa |
| GSGTDFTLTISSLQVEDVAVYYCQQYYTTPKTFGQGTKV | |
| (SEQ ID NO: 141) | |
| MKQSTIALALLPLLFTPVTKAEIVLTQSPTTLSLSPGERATLSCR | SEQ ID NO: 36 |
| ASQSVGRYLAWYQQRPGQAPRLLVFDTSNRAPGVPARFSGRG | −1 C-term aa |
| SGTLFTLTISSLEPEDSAVYFCQQRSSGLTFGGGTKVTV | |
| (SEQ ID NO: 142) | |
| MKQSTIALALLPLLFTPVTKAEIVLTQSPTTLSLSPGERATLSCR | SEQ ID NO: 36 |
| ASQSVGRYLAWYQQRPGQAPRLLVFDTSNRAPGVPARFSGRG | −2 C-term aa |
| SGTLFTLTISSLEPEDSAVYFCQQRSSGLTFGGGTKVT | |
| (SEQ ID NO: 143) | |
| MKQSTIALALLPLLFTPVTKAEIVLTQSPTTLSLSPGERATLSCR | SEQ ID NO: 36 |
| ASQSVGRYLAWYQQRPGQAPRLLVFDTSNRAPGVPARFSGRG | −3 C-term aa |
| SGTLFTLTISSLEPEDSAVYFCQQRSSGLTFGGGTKV | |
| (SEQ ID NO: 144) | |
| MKQSTIALALLPLLFTPVTKAEIVLTQSPTTLSLSPGERATLSCR | SEQ ID NO: 46 |
| ASQSVGRYLAWYQQRPGQAPRLLCFDTSNRAPGVPARFSCRG | −1 C-term aa |
| SGTLFTLTISSLEPEDSAVYFCQQRSSGLTFGGGTKVTV | |
| (SEQ ID NO: 145) | |
| MKQSTIALALLPLLFTPVTKAEIVLTQSPTTLSLSPGERATLSCR | SEQ ID NO: 46 |
| ASQSVGRYLAWYQQRPGQAPRLLCFDTSNRAPGVPARFSCRG | −2 C-term aa |
| SGTLFTLTISSLEPEDSAVYFCQQRSSGLTFGGGTKVT | |
| (SEQ ID NO: 146) | |
| MKQSTIALALLPLLFTPVTKAEIVLTQSPTTLSLSPGERATLSCR | SEQ ID NO: 46 |
| ASQSVGRYLAWYQQRPGQAPRLLCFDTSNRAPGVPARFSCRG | −3 C-term aa |
| SGTLFTLTISSLEPEDSAVYFCQQRSSGLTFGGGTKV | |
| (SEQ ID NO: 147) | |
| MKQSTIALALLPLLFTPVTKAEIVMTQSPVTLSLSPGERATLSC | SEQ ID NO: 56 |
| RASQSVGTSLAWYQQKPGQAPRLLIYDASNRATGISARFSGSG | −1 C-term aa |
| SGTDFTLTISSLEPEDFAVYYCQQRYNWPRTFGGGTKVTV | |
| (SEQ ID NO: 148) | |
| MKQSTIALALLPLLFTPVTKAEIVMTQSPVTLSLSPGERATLSC | SEQ ID NO: 56 |
| RASQSVGTSLAWYQQKPGQAPRLLIYDASNRATGISARFSGSG | −2 C-term aa |
| SGTDFTLTISSLEPEDFAVYYCQQRYNWPRTFGGGTKVT | |
| (SEQ ID NO: 149) | |
| MKQSTIALALLPLLFTPVTKAEIVMTQSPVTLSLSPGERATLSC | SEQ ID NO: 56 |
| RASQSVGTSLAWYQQKPGQAPRLLIYDASNRATGISARFSGSG | −3 C-term aa |
| SGTDFTLTISSLEPEDFAVYYCQQRYNWPRTFGGGTKV | |
| (SEQ ID NO: 150) | |
| MKQSTIALALLPLLFTPVTKAEIVMTQSPVTLSLSPGERATLSC | SEQ ID NO: 66 |
| RASQSVGTSLAWYQQKPGQAPRLLCYDASNRATGISARFSCS | −1 C-term aa |
| GSGTDFTLTISSLEPEDFAVYYCQQRYNWPRTFGGGTKVTV | |
| (SEQ ID NO: 151) | |
| MKQSTIALALLPLLFTPVTKAEIVMTQSPVTLSLSPGERATLSC | SEQ ID NO: 66 |
| RASQSVGTSLAWYQQKPGQAPRLLCYDASNRATGISARFSCS | −2 C-term aa |
| GSGTDFTLTISSLEPEDFAVYYCQQRYNWPRTFGGGTKVT | |
| (SEQ ID NO: 152) | |
| MKQSTIALALLPLLFTPVTKAEIVMTQSPVTLSLSPGERATLSC | SEQ ID NO: 66 |
| RASQSVGTSLAWYQQKPGQAPRLLCYDASNRATGISARFSCS | −3 C-term aa |
| GSGTDFTLTISSLEPEDFAVYYCQQRYNWPRTFGGGTKV | |
| (SEQ ID NO: 153) | |
| MKQSTIALALLPLLFTPVTKAQVQLVQSGGGVVQPGRSLRLSC | SEQ ID NO: 76 |
| AASGFIVDGYAMHWVRQAPGQGLEWVSVTNNGGSTSYADSV | −1 C-term aa |
| KGRFTISRDNSKNTVYLQMNSLRAEDTAVYYCARQSITGPTG | |
| AFDIWGQGTMVTVS (SEQ ID NO: 154) | |
| MKQSTIALALLPLLFTPVTKAQVQLVQSGGGVVQPGRSLRLSC | SEQ ID NO: 76 |
| AASGFIVDGYAMHWVRQAPGQGLEWVSVTNNGGSTSYADSV | −2 C-term aa |
| KGRFTISRDNSKNTVYLQMNSLRAEDTAVYYCARQSITGPTG | |
| AFDIWGQGTMVTV (SEQ ID NO: 155) | |
| MKQSTIALALLPLLFTPVTKAQVQLVQSGGGVVQPGRSLRLSC | SEQ ID NO: 76 |
| AASGFIVDGYAMHWVRQAPGQGLEWVSVTNNGGSTSYADSV | −3 C-term aa |
| KGRFTISRDNSKNTVYLQMNSLRAEDTAVYYCARQSITGPTG | |
| AFDIWGQGTMVT (SEQ ID NO: 156) | |
| MKQSTIALALLPLLFTPVTKAQVQLVQSGGGVVQPGRSLRLSC | SEQ ID NO: 86 |
| AASGFIVDGYAMHWVRQAPGQGLEWVCVTNNGGSTSYADS | −1 C-term aa |
| VKGRFTCSRDNSKNTVYLQMNSLRAEDTAVYYCARQSITGPT | |
| GAFDIWGQGTMVTVS (SEQ ID NO: 157) | |
| MKQSTIALALLPLLFTPVTKAQVQLVQSGGGVVQPGRSLRLSC | SEQ ID NO: 86 |
| AASGFIVDGYAMHWVRQAPGQGLEWVCVTNNGGSTSYADS | −2 C-term aa |
| VKGRFTCSRDNSKNTVYLQMNSLRAEDTAVYYCARQSITGPT | |
| GAFDIWGQGTMVTV (SEQ ID NO: 158) | |
| MKQSTIALALLPLLFTPVTKAQVQLVQSGGGVVQPGRSLRLSC | SEQ ID NO: 86 |
| AASGFIVDGYAMHWVRQAPGQGLEWVCVTNNGGSTSYADS | −3 C-term aa |
| VKGRFTCSRDNSKNTVYLQMNSLRAEDTAVYYCARQSITGPT | |
| GAFDIWGQGTMVT (SEQ ID NO: 159) | |
| MKQSTIALALLPLLFTPVTKAQVQLVESGGGLVKPGGSLRLSC | SEQ ID NO: 96 |
| AASGFTFSNAWMTWVRQAPGKGLEWVGRIKTKTDGGTTDYA | −1 C-term aa |
| APVKGRFTISRDDSKNTLYLQMNSLKTEDTAVYYCTTDRDHS | |
| SGSWGQGTLVTVS (SEQ ID NO: 160) | |
| MKQSTIALALLPLLFTPVTKAQVQLVESGGGLVKPGGSLRLSC | SEQ ID NO: 96 |
| AASGFTFSNAWMTWVRQAPGKGLEWVGRIKTKTDGGTTDYA | −2 C-term aa |
| APVKGRFTISRDDSKNTLYLQMNSLKTEDTAVYYCTTDRDHS | |
| SGSWGQGTLVTV (SEQ ID NO: 161) | |
| MKQSTIALALLPLLFTPVTKAQVQLVESGGGLVKPGGSLRLSC | SEQ ID NO: 96 |
| AASGFTFSNAWMTWVRQAPGKGLEWVGRIKTKTDGGTTDYA | −3 C-term aa |
| APVKGRFTISRDDSKNTLYLQMNSLKTEDTAVYYCTTDRDHS | |
| SGSWGQGTLVT (SEQ ID NO: 162) | |
| MKQSTIALALLPLLFTPVTKAEVQLVQSGGGLVQPGRSLRLSC | SEQ ID NO: 106 |
| AASGFTFDDYAMHWVRQAPGKGLEWVSGISGSGASTYYADS | −1 C-term aa |
| VKGRFTISRDNSKNTLYLQMNSLRAGDTALYYCARQSITGPTG | |
| AFDVWGQGTMVTVS (SEQ ID NO: 163) | |
| MKQSTIALALLPLLFTPVTKAEVQLVQSGGGLVQPGRSLRLSC | SEQ ID NO: 106 |
| AASGFTFDDYAMHWVRQAPGKGLEWVSGISGSGASTYYADS | −2 C-term aa |
| VKGRFTISRDNSKNTLYLQMNSLRAGDTALYYCARQSITGPTG | |
| AFDVWGQGTMVTV (SEQ ID NO: 164) | |
| MKQSTIALALLPLLFTPVTKAEVQLVQSGGGLVQPGRSLRLSC | SEQ ID NO: 106 |
| AASGFTFDDYAMHWVRQAPGKGLEWVSGISGSGASTYYADS | −3 C-term aa |
| VKGRFTISRDNSKNTLYLQMNSLRAGDTALYYCARQSITGPTG | |
| AFDVWGQGTMVT (SEQ ID NO: 165) | |
| MKQSTIALALLPLLFTPVTKAQLQLQESGGGVVQPGRSLRLSC | SEQ ID NO: 116 |
| AASGFTFSSYAMSWVRQAPGKGLEWVCAISGSGGSTYYADSV | −1 C-term aa |
| KGRFTCSRDNSKNTLYLQMNSLRAEDTAVYYCAKDGKGGSS | |
| GYDHPDYWGQGTLVTVS (SEQ ID NO: 166) | |
| MKQSTIALALLPLLFTPVTKAQLQLQESGGGVVQPGRSLRLSC | SEQ ID NO: 116 |
| AASGFTFSSYAMSWVRQAPGKGLEWVCAISGSGGSTYYADSV | −2 C-term aa |
| KGRFTCSRDNSKNTLYLQMNSLRAEDTAVYYCAKDGKGGSS | |
| GYDHPDYWGQGTLVTV (SEQ ID NO: 167) | |
| MKQSTIALALLPLLFTPVTKAQLQLQESGGGVVQPGRSLRLSC | SEQ ID NO: 116 |
| AASGFTFSSYAMSWVRQAPGKGLEWVCAISGSGGSTYYADSV | −3 C-term aa |
| KGRFTCSRDNSKNTLYLQMNSLRAEDTAVYYCAKDGKGGSS | |
| GYDHPDYWGQGTLVT (SEQ ID NO: 168) | |
| MKQSTIALALLPLLFTPVTKAQVQLVESGGGLIKPGGSLRLSC | SEQ ID NO: 126 |
| AASGFTFSNYAMSWVRQAPGKGLEWVCAISSSGGSTYYADSV | −1 C-term aa |
| KGRFTCSRDNSKNTVYLQMNSLRAEDTAVYYCVREEYRCSGT | |
| SCPGAFDIWGQGTMVTVS (SEQ ID NO: 169) | |
| MKQSTIALALLPLLFTPVTKAQVQLVESGGGLIKPGGSLRLSC | SEQ ID NO: 126 |
| AASGFTFSNYAMSWVRQAPGKGLEWVCAISSSGGSTYYADSV | −2 C-term aa |
| KGRFTCSRDNSKNTVYLQMNSLRAEDTAVYYCVREEYRCSGT | |
| SCPGAFDIWGQGTMVTV (SEQ ID NO: 170) | |
| MKQSTIALALLPLLFTPVTKAQVQLVESGGGLIKPGGSLRLSC | SEQ ID NO: 126 |
| AASGFTFSNYAMSWVRQAPGKGLEWVCAISSSGGSTYYADSV | −3 C-term aa |
| KGRFTCSRDNSKNTVYLQMNSLRAEDTAVYYCVREEYRCSGT | |
| SCPGAFDIWGQGTMVT (SEQ ID NO: 171) | |

The data in FIGS. 4-9 clearly show the presence of pre-existing antibody response to a number of wild type V_{H} single domain scaffolds. Furthermore, the data surprisingly demonstrates the complete elimination, or a significant reduction, of the pre-existing antibody response with the addition of one or more amino acid residues to the C-terminus of each of the different V_{H} single domain scaffolds only. The same pattern of response is repeatedly seen with each V_{H} scaffold, although there is a tendency for an increased reduction in response for the Ala-Ser-Thr amino acid addition compared with other additions. This demonstrates that modification at the C-terminus of the V_{H} single domain scaffold could eliminate the interaction of the pre-existing antibody with the scaffold by altering the three dimensional configuration of the C-terminal single domain antibody such that the pre-existing antibody no longer recognizes the single domain antibody; may alter the exposure of the C-terminal single domain antibody to the pre-existing antibody such that it does not react; alters the steric hindrance between the single domain antibody and the pre-existing antibody; disrupts at least one conformational neoepitope in the C-terminus, or shields at least one neoepitope in framework of the single domain antibody.

Collectively, this data shows for the first time that there is a neoepitope in the C-terminus of several human V_{H} single domain scaffolds, which is recognized by preexisting antibodies present in the sera of healthy human volunteers. Identification of the preexisting immune response to these human V_{H} single domain scaffolds was an unexpected and surprising observation. Moreover, modification to the C-terminus of the V_{H} by either addition or deletion of amino acids, eliminated the preexisting antibody response.

1. An isolated V_{H} single domain antibody comprising a C-terminal modification, wherein the C-terminal modification comprises a deletion of at least one amino acid residue such that the deletion of at least one amino acid residue in the single domain antibody eliminates the interaction of a pre-existing antibody with the single domain antibody without interfering with the binding of the single domain antibody with its target.

2. The isolated single domain antibody of claim 1, wherein the C-terminal of the single domain antibody is exposed such that the exposed C-terminal is available for interaction with the pre-existing antibody, and wherein the C-terminal modification reduces the exposure of the C-terminal to the pre-existing antibody.

3. The isolated single domain antibody of claim 1, wherein the C-terminal modification modifies the C-terminus of the single domain antibody by a mechanism selected from the group consisting of eliminating the interaction of the pre-existing antibody by altering the three dimensional configuration of the C-terminal single domain antibody such that the pre-existing antibody no longer recognizes the single domain antibody, alters the exposure of the C-terminal single domain antibody to the pre-existing antibody, alters the steric hindrance between the single domain antibody and the pre-existing antibody, disrupts at least one conformational neoepitope in the C-terminus, and shields at least one neoepitope in a framework of the single domain antibody.

4. The isolated single domain antibody of claim 1, wherein the single domain antibody is a human V_{H}.

5. The isolated single domain antibody of claim 4, wherein the human V_{H} is selected from the group consisting of SEQ ID NO: 8, SEQ ID NO: 9, SEQ ID NO: 10, SEQ ID NO: 11, SEQ ID NO: 12, and SEQ ID NO: 13.

6. The isolated single domain antibody of claim 1, wherein the amino acid residue is selected from the group consisting of a naturally occurring amino acid or a non-naturally occurring amino acid.

7. The isolated single domain antibody of claim 1, wherein the C-terminal modification further comprises the deletion of at least one additional amino acid residue from the C-terminus of the single domain antibody.

8. The isolated single domain antibody of claim 1, wherein the C-terminal modification further comprises the deletion of at least two additional amino acid residues from the C-terminus of the single domain antibody.

9. The isolated single domain antibody of claim 1, wherein the C-terminal modification further comprises the deletion of at least three, at least four, at least five, at least six, at least seven, at least eight, at least nine additional amino acid residues from the C-terminus of the single domain antibody.

10. The isolated single domain antibody of claim 1, wherein the C-terminal modification is the deletion of an amino acid sequence selected from the group consisting of Arg, Arg-Thr, Arg-Thr-Val, Gly-Gln, and Gly-Gln-Pro.

11. The isolated single domain antibody of claim 1, wherein the C-terminal modification eliminates a pre-existing antibody response at least by about 10% compared with an single domain antibody without the C-terminal modification.

12. The isolated single domain antibody of claim 1, wherein the V_{H} comprises SEQ ID NO: 8, and wherein the C-terminal modification comprises a deletion of one amino acid to three amino acids.

13. The isolated single domain antibody of claim 1, wherein the V_{H} comprises SEQ ID NO: 9, and wherein the C-terminal modification comprises a deletion of one amino acid to three amino acids.

14. The isolated single domain antibody of claim 1, wherein the V_{H} comprises SEQ ID NO: 10, and wherein the C-terminal modification comprises a deletion of one amino acid to three amino acids.

15. The isolated single domain antibody of claim 1, wherein the V_{H} comprises SEQ ID NO: 11, and wherein the C-terminal modification comprises a deletion of one amino acid to three amino acids.

16. The isolated single domain antibody of claim 1, wherein the V_{H} comprises SEQ ID NO: 12, and wherein the C-terminal modification comprises a deletion of one amino acid to three amino acids.

17. The isolated single domain antibody of claim 1, wherein the V_{H} comprises SEQ ID NO: 13, and wherein the C-terminal modification comprises a deletion of one amino acid to three amino acids.

18. A nucleic acid encoding a composition of claim 1.

19. An expression vector comprising the nucleic acid of claim 18.

20. A host cell or organism comprising the expression vector of claim 19.

21. A pharmaceutical composition comprising the single domain antibody of claim 1.

22. A method of eliminating a pre-existing immune response in a subject comprising: administering a single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target.

23. A method of improving a response to single domain antibody in a subject having a pre-existing antibody against a single domain antibody, comprising: administering single domain antibody comprising a C-terminal modification, wherein the C-terminal modification comprises a deletion of at least one amino acid residue such that the deletion of at least one amino acid residue to the single domain antibody eliminates the interaction of a pre-existing antibody with the single domain antibody without interfering with the binding of the single domain antibody with its target.

24. A method of predicting whether a pre-existing antibody will produce a pre-existing immune response with a single domain antibody comprising:
contacting the single domain antibody with a human sample;
determining whether a pre-existing antibody, if present in the human sample, binds to the single domain scaffold; and
modifying the C-terminal region of single domain antibody by the deletion of at least one amino acid residue such that the C-terminal modification eliminates the interaction of the pre-existing antibody with the single domain antibody.

25. The method of claim 18, wherein the human sample is selected from the group consisting of blood and serum.


SEQUENCE LISTING (173 sequences)

SEQ ID NO: 1 - protein, 21 aa, Artificial Sequence
  Description of Artificial Sequence: Synthetic peptide
MKQSTIALALLPLLFTPVTKA

SEQ ID NO: 2 - protein, 108 aa, Homo sapiens
EIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIK

SEQ ID NO: 3 - protein, 107 aa, Homo sapiens
ETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIK

SEQ ID NO: 4 - protein, 106 aa, Homo sapiens
EIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVL

SEQ ID NO: 5 - protein, 106 aa, Homo sapiens
EIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVL

SEQ ID NO: 6 - protein, 107 aa, Homo sapiens
EIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVL

SEQ ID NO: 7 - protein, 107 aa, Homo sapiens
EIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVL

SEQ ID NO: 8 - protein, 120 aa, Homo sapiens
QVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSS

SEQ ID NO: 9 - protein, 120 aa, Homo sapiens
QVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSS

SEQ ID NO: 10 - protein, 119 aa, Homo sapiens
QVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSS

SEQ ID NO: 11 - protein, 121 aa, Homo sapiens
EVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSS

SEQ ID NO: 12 - protein, 123 aa, Homo sapiens
QLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSS

SEQ ID NO: 13 - protein, 125 aa, Homo sapiens
QVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSS

SEQ ID NO: 14 - protein, 5 aa, Artificial Sequence
  Description of Artificial Sequence: Synthetic peptide
ASTKP

SEQ ID NO: 15 - protein, 5 aa, Artificial Sequence
  Description of Artificial Sequence: Synthetic peptide
GGGGS

SEQ ID NO: 16 - protein, 129 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIK

SEQ ID NO: 17 - protein, 130 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKA

SEQ ID NO: 18 - protein, 131 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKAA

SEQ ID NO: 19 - protein, 131 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKAS

SEQ ID NO: 20 - protein, 132 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKAST

SEQ ID NO: 21 - protein, 134 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKASTKP

SEQ ID NO: 22 - protein, 134 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKGGGGS

SEQ ID NO: 23 - protein, 130 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKG

SEQ ID NO: 24 - protein, 131 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKGG

SEQ ID NO: 25 - protein, 131 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIKGS

SEQ ID NO: 26 - protein, 128 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIK

SEQ ID NO: 27 - protein, 129 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKA

SEQ ID NO: 28 - protein, 130 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKAA

SEQ ID NO: 29 - protein, 130 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKAS

SEQ ID NO: 30 - protein, 131 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKAST

SEQ ID NO: 31 - protein, 133 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKASTKP

SEQ ID NO: 32 - protein, 133 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKGGGGS

SEQ ID NO: 33 - protein, 129 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKG

SEQ ID NO: 34 - protein, 130 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKGG

SEQ ID NO: 35 - protein, 130 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEIKGS

SEQ ID NO: 36 - protein, 127 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVL

SEQ ID NO: 37 - protein, 128 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLA

SEQ ID NO: 38 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLAA

SEQ ID NO: 39 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLAS

SEQ ID NO: 40 - protein, 130 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLAST

SEQ ID NO: 41 - protein, 132 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLASTKP

SEQ ID NO: 42 - protein, 132 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLGGGGS

SEQ ID NO: 43 - protein, 128 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLG

SEQ ID NO: 44 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLGG

SEQ ID NO: 45 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVLGS

SEQ ID NO: 46 - protein, 127 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVL

SEQ ID NO: 47 - protein, 128 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLA

SEQ ID NO: 48 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLAA

SEQ ID NO: 49 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLAS

SEQ ID NO: 50 - protein, 130 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLAST

SEQ ID NO: 51 - protein, 132 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLASTKP

SEQ ID NO: 52 - protein, 132 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLGGGGS

SEQ ID NO: 53 - protein, 128 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLG

SEQ ID NO: 54 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLGG

SEQ ID NO: 55 - protein, 129 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTVLGS

SEQ ID NO: 56 - protein, 128 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVL

SEQ ID NO: 57 - protein, 129 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLA

SEQ ID NO: 58 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLAA

SEQ ID NO: 59 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLAS

SEQ ID NO: 60 - protein, 131 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLAST

SEQ ID NO: 61 - protein, 133 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLASTKP

SEQ ID NO: 62 - protein, 133 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLGGGGS

SEQ ID NO: 63 - protein, 129 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLG

SEQ ID NO: 64 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLGG

SEQ ID NO: 65 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVLGS

SEQ ID NO: 66 - protein, 128 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVL

SEQ ID NO: 67 - protein, 129 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLA

SEQ ID NO: 68 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLAA

SEQ ID NO: 69 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLAS

SEQ ID NO: 70 - protein, 131 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLAST

SEQ ID NO: 71 - protein, 133 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLASTKP

SEQ ID NO: 72 - protein, 133 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLGGGGS

SEQ ID NO: 73 - protein, 129 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLG

SEQ ID NO: 74 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLGG

SEQ ID NO: 75 - protein, 130 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTVLGS

SEQ ID NO: 76 - protein, 141 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSS

SEQ ID NO: 77 - protein, 142 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSA

SEQ ID NO: 78 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSAA

SEQ ID NO: 79 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSAS

SEQ ID NO: 80 - protein, 144 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSAST

SEQ ID NO: 81 - protein, 146 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSASTKP

SEQ ID NO: 82 - protein, 146 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSGGGGS

SEQ ID NO: 83 - protein, 142 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSG

SEQ ID NO: 84 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSGG

SEQ ID NO: 85 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVSSGS

SEQ ID NO: 86 - protein, 141 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSS

SEQ ID NO: 87 - protein, 142 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSA

SEQ ID NO: 88 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSAA

SEQ ID NO: 89 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSAS

SEQ ID NO: 90 - protein, 144 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSAST

SEQ ID NO: 91 - protein, 146 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSASTKP

SEQ ID NO: 92 - protein, 146 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSGGGGS

SEQ ID NO: 93 - protein, 142 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSG

SEQ ID NO: 94 - skipped

SEQ ID NO: 95 - protein, 143 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVSSGS

SEQ ID NO: 96 - protein, 140 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSS

SEQ ID NO: 97 - protein, 141 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSA

SEQ ID NO: 98 - protein, 142 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSAA

SEQ ID NO: 99 - protein, 142 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSAS

SEQ ID NO: 100 - protein, 143 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSAST

SEQ ID NO: 101 - protein, 145 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSASTKP

SEQ ID NO: 102 - protein, 145 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSGGGGS

SEQ ID NO: 103 - protein, 141 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSG

SEQ ID NO: 104 - protein, 142 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSGG

SEQ ID NO: 105 - protein, 142 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSSGS

SEQ ID NO: 106 - protein, 142 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSS

SEQ ID NO: 107 - protein, 143 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSA

SEQ ID NO: 108 - protein, 144 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSAA

SEQ ID NO: 109 - protein, 144 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSAS

SEQ ID NO: 110 - protein, 145 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSAST

SEQ ID NO: 111 - protein, 147 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSASTKP

SEQ ID NO: 112 - protein, 147 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSGGGGS

SEQ ID NO: 113 - protein, 143 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSG

SEQ ID NO: 114 - protein, 144 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSGG

SEQ ID NO: 115 - protein, 144 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSSGS

SEQ ID NO: 116 - protein, 144 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSS

SEQ ID NO: 117 - protein, 145 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSA

SEQ ID NO: 118 - protein, 146 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSAA

SEQ ID NO: 119 - protein, 146 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSAS

SEQ ID NO: 120 - protein, 147 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSAST

SEQ ID NO: 121 - protein, 149 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSASTKP

SEQ ID NO: 122 - protein, 149 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSGGGGS

SEQ ID NO: 123 - protein, 145 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSG

SEQ ID NO: 124 - protein, 146 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSGG

SEQ ID NO: 125 - protein, 146 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVSSGS

SEQ ID NO: 126 - protein, 146 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSS

SEQ ID NO: 127 - protein, 147 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSA

SEQ ID NO: 128 - protein, 148 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSAA

SEQ ID NO: 129 - protein, 148 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSAS

SEQ ID NO: 130 - protein, 149 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSAST

SEQ ID NO: 131 - protein, 151 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSASTKP

SEQ ID NO: 132 - protein, 151 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSGGGGS

SEQ ID NO: 133 - protein, 147 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSG

SEQ ID NO: 134 - protein, 148 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSGG

SEQ ID NO: 135 - protein, 148 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVSSGS

SEQ ID NO: 136 - protein, 128 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEI

SEQ ID NO: 137 - protein, 127 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVE

SEQ ID NO: 138 - protein, 126 aa, Artificial Sequence
  Description of Artificial Sequence: Synthetic polypeptide
MKQSTIALALLPLLFTPVTKAEIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKV

SEQ ID NO: 139 - protein, 127 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVEI

SEQ ID NO: 140 - protein, 126 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKVE

SEQ ID NO: 141 - protein, 125 aa, Artificial Sequence
  Description of Artificial Sequence: Synthetic polypeptide
MKQSTIALALLPLLFTPVTKAETTLTQSPGTLSLSPGERATLSCRASQSVRNNLAWYQQRPGQAPRLLCYGASTRATGIPARFSCSGSGTDFTLTISSLQVEDVAVYYCQQYYTTPKTFGQGTKV

SEQ ID NO: 142 - protein, 126 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTV

SEQ ID NO: 143 - protein, 125 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVT

SEQ ID NO: 144 - protein, 124 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKV

SEQ ID NO: 145 - protein, 126 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVTV

SEQ ID NO: 146 - protein, 125 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKVT

SEQ ID NO: 147 - protein, 124 aa, Artificial Sequence
  Description of Artificial Sequence: Synthetic polypeptide
MKQSTIALALLPLLFTPVTKAEIVLTQSPTTLSLSPGERATLSCRASQSVGRYLAWYQQRPGQAPRLLCFDTSNRAPGVPARFSCRGSGTLFTLTISSLEPEDSAVYFCQQRSSGLTFGGGTKV

SEQ ID NO: 148 - protein, 127 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTV

SEQ ID NO: 149 - protein, 126 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVT

SEQ ID NO: 150 - protein, 125 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKV

SEQ ID NO: 151 - protein, 127 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVTV

SEQ ID NO: 152 - protein, 126 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKVT

SEQ ID NO: 153 - protein, 125 aa, Artificial Sequence
  Description of Artificial Sequence: Synthetic polypeptide
MKQSTIALALLPLLFTPVTKAEIVMTQSPVTLSLSPGERATLSCRASQSVGTSLAWYQQKPGQAPRLLCYDASNRATGISARFSCSGSGTDFTLTISSLEPEDFAVYYCQQRYNWPRTFGGGTKV

SEQ ID NO: 154 - protein, 140 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTVS

SEQ ID NO: 155 - protein, 139 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVTV

SEQ ID NO: 156 - protein, 138 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVSVTNNGGSTSYADSVKGRFTISRDNSKNTVYLQMNSLRAEDTAVYYCARQSITGPTGAFDIWGQGTMVT

SEQ ID NO: 157 - protein, 140 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTVS

SEQ ID NO: 158 - protein, 139 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVTV

SEQ ID NO: 159 - protein, 138 aa, Artificial Sequence
  Description of Artificial Sequence: Synthetic polypeptide
MKQSTIALALLPLLFTPVTKAQVQLVQSGGGVVQPGRSLRLSCAASGFIVDGYAMHWVRQAPGQGLEWVCVTNNGGSTSYADSVKGRFTCSRDNSKNTVYLQMNSLRAEDTAVYYCARQSITGPTGAFDIWGQGTMVT

SEQ ID NO: 160 - protein, 139 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVS

SEQ ID NO: 161 - protein, 138 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTV

SEQ ID NO: 162 - protein, 137 aa, Artificial Sequence
  Description of Artificial Sequence: Synthetic polypeptide
MKQSTIALALLPLLFTPVTKAQVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVT

SEQ ID NO: 163 - protein, 141 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVS

SEQ ID NO: 164 - protein, 140 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTV

SEQ ID NO: 165 - protein, 139 aa, Artificial Sequence
  Description of Artificial Sequence: Synthetic polypeptide
MKQSTIALALLPLLFTPVTKAEVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVT

SEQ ID NO: 166 - protein, 143 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTVS

SEQ ID NO: 167 - protein, 142 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVTV

SEQ ID NO: 168 - protein, 141 aa, Artificial Sequence
  Description of Artificial Sequence: Synthetic polypeptide
MKQSTIALALLPLLFTPVTKAQLQLQESGGGVVQPGRSLRLSCAASGFTFSSYAMSWVRQAPGKGLEWVCAISGSGGSTYYADSVKGRFTCSRDNSKNTLYLQMNSLRAEDTAVYYCAKDGKGGSSGYDHPDYWGQGTLVT

SEQ ID NO: 169 - protein, 145 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTVS

SEQ ID NO: 170 - protein, 144 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVTV

SEQ ID NO: 171 - protein, 143 aa, Artificial Sequence
  Description of Artificial Sequence: Synthetic polypeptide
MKQSTIALALLPLLFTPVTKAQVQLVESGGGLIKPGGSLRLSCAASGFTFSNYAMSWVRQAPGKGLEWVCAISSSGGSTYYADSVKGRFTCSRDNSKNTVYLQMNSLRAEDTAVYYCVREEYRCSGTSCPGAFDIWGQGTMVT

SEQ ID NO: 172 - protein, 6 aa, Artificial Sequence
  Description of Artificial Sequence: Synthetic peptide
TKVEIK

SEQ ID NO: 173 - protein, 6 aa, Artificial Sequence
  Description of Artificial Sequence: Synthetic peptide
TKVTVL